SEQUENCE LISTING (44 sequences)

SEQ ID NO: 1 - protein, 400 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALGQLTGGYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLAESNSADDETDDPRRYQKYGYIGTHEYPHFSHRPSTLQAASTPQAEEDWTCTIL

SEQ ID NO: 2 - protein, 364 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALGQLTGGYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVRPPASSLPFIPAPLHEA

SEQ ID NO: 3 - protein, 414 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALGQLTGGYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLTQHTPGSIHPTGRRGLDLHHPLNASASWGKGLQCYLDQFLHFQVGLLIQRGQSSSVSWCIIQDRTQVS

SEQ ID NO: 4 - protein, 360 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALGQLTGGYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVNLTLVGRRNYTNN

SEQ ID NO: 5 - protein, 406 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALAHRGVHRPAERGRVLHLLHRTTERLPEAAPHQAQEPHPPSQALVPKCMALPPGLCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLTQHTPGSIHPTGRRGLDLHHPLNASASWGKGLQCYLDQFLHFQVGLLIQRGQSSSVSWCIIQDRTQVS

SEQ ID NO: 6 - protein, 392 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALAHRGVHRPAERGRVLHLLHRTTERLPEAAPHQAQEPHPPSQALVPKCMALPPGLCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLAESNSADDETDDPRRYQKYGYIGTHEYPHFSHRPSTLQAASTPQAEEDWTCTIL

SEQ ID NO: 7 - protein, 384 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALGQLTGGYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLMRQRLREVRSLAQGHQLTSGGNGIQAQWTLKPVLMSLC

SEQ ID NO: 8 - protein, 376 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALAHRGVHRPAERGRVLHLLHRTTERLPEAAPHQAQEPHPPSQALVPKCMALPPGLCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLMRQRLREVRSLAQGHQLTSGGNGIQAQWTLKPVLMSLC

SEQ ID NO: 9 - protein, 356 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALAHRGVHRPAERGRVLHLLHRTTERLPEAAPHQAQEPHPPSQALVPKCMALPPGLCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVRPPASSLPFIPAPLHEA

SEQ ID NO: 10 - protein, 352 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALAHRGVHRPAERGRVLHLLHRTTERLPEAAPHQAQEPHPPSQALVPKCMALPPGLCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVNLTLVGRRNYTNN

SEQ ID NO: 11 - protein, 256 aa, Homo sapiens
MMDLRNTPAKSLDKFIEDYLLPDTCFRMQINHAIDIICGFLKERCFRGSSYPVCVSKVVKCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLTQHTPGSIHPTGRRGLDLHHPLNASASWGKGLQCYLDQFLHFQVGLLIQRGQSSSVSWCIIQDRTQVS

SEQ ID NO: 12 - protein, 242 aa, Homo sapiens
MMDLRNTPAKSLDKFIEDYLLPDTCFRMQINHAIDIICGFLKERCFRGSSYPVCVSKVVKCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLAESNSADDETDDPRRYQKYGYIGTHEYPHFSHRPSTLQAASTPQAEEDWTCTIL

SEQ ID NO: 13 - protein, 226 aa, Homo sapiens
MMDLRNTPAKSLDKFIEDYLLPDTCFRMQINHAIDIICGFLKERCFRGSSYPVCVSKVVKCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLMRQRLREVRSLAQGHQLTSGGNGIQAQWTLKPVLMSLC

SEQ ID NO: 14 - protein, 206 aa, Homo sapiens
MMDLRNTPAKSLDKFIEDYLLPDTCFRMQINHAIDIICGFLKERCFRGSSYPVCVSKVVKCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVRPPASSLPFIPAPLHEA

SEQ ID NO: 15 - protein, 382 aa, Homo sapiens
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALGQLTGSYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVNLTLVGRRNYPIISEHAVNLQQTRRASLSYSFQVA

SEQ ID NO: 16 - protein, 365 aa, synthetic construct
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALGQLTGSYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVRPPASSLPFIPAPLHEAL

SEQ ID NO: 17 - protein, 346 aa, synthetic construct
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQRERAFSVKFEVQAPRWGNPRALSFVLSSLQLGEGVEFDVLPAFDALGQLTGSYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILL

SEQ ID NO: 18 - protein, 364 aa, synthetic construct
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQSERAFSVKFEVQAPRWGNPRALSFVLSSLQLGEGVEFDVLPAFDALGQLTGGYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVRPPASSLPFIPAPLHEA

SEQ ID NO: 19 - protein, 364 aa, synthetic construct [sequence omitted]

SEQ ID NO: 20 - protein, 364 aa, synthetic construct
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQSERAFSVKFEVQAPRWGNPRALSFVLSSLQLGEGVEFDVLPAFDALGQLTGSYKPNPQIYVKLIEECTDLQKEGEFSTCFTELQRDFLKQRPTKLKSLIRLVKHWYQNCKKKLGKLPPQYALELLTVYAWERGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVRPPASSLPFIPAPLHEA

SEQ ID NO: 21 - protein, 364 aa, synthetic construct
MMDLRNTPAKSLDKFIEDYLLPDTCFRMQINHAIDIICGFLKERCFRGSSYPVCVSKVVKGGSSGKGTTLRGRSDADLVVFLSPLTTFQDQLNRRGEFIQEIRRQLEACQSERAFSVKFEVQAPRWGNPRALSFVLSSLQLGEGVEFDVLPAFDALGQLTGSYKPNPQIYVKLIEECTDLQKEGEFSTCFTELQRDFLKQRPTKLKSLIRLVKHWYQNCKKKLGKLPPQYALELLTVYAWEQGSMKTHFNTAQGFRTVLELVINYQQLCIYWTKYYDFKNPIIEKYLRRQLTKPRPVILDPADPTGNLGGGDPKGWRQLAQEAEAWLNYPCFKNWDGSPVSSWILLVRPPASSLPFIPAPLHEA

SEQ ID NO: 22 - protein, 687 aa, synthetic construct
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPVKVI

SEQ ID NO: 23 - protein, 719 aa, synthetic construct
MGNGESQLSSVPAQKLGWFIQEYLKPYEECQTLIDEMVNTICDVLQEPEQFPLVQGVAIGGSYGRKTVLRGNSDGTLVLFFSDLKQFQDQKRSQRDILDKTGDKLKFCLFTKWLKNNFEIQKSLDGFTIQVFTKNQRISFEVLAAFNALSLNDNPSPWIYRELKRSLDKTNASPGEFAVCFTELQQKFFDNRPGKLKDLILLIKHWHQQCQKKIKDLPSLSPYALELLTVYAWEQGCRKDNFDIAEGVRTVLELIKCQEKLCIYWMVNYNFEDETIRNILLHQLQSARPVILDPVDPTNNVSGDKICWQWLKKEAQTWLTSPNLDNELPAPSWNVLPAPLFTTPGHLLDKFIKEFLQPNKCFLEQIDSAVNIIRTFLKENCFRQSTAKIQIVRGGSTAKGTALKTGSDADLVVFHNSLKSYTSQKNERHKIVKEIHEQLKAFWREKEEELEVSFEPPKWKAPRVLSFSLKSKVLNESVSFDVLPAFNALGQLSSGSTPSPEVYAGLIDLYKSSDLPGGEFSTCFTVLQRNFIRSRPTKLKDLIRLVKHWYKECERKLKPKGSLPPKYALELLTIYAWEQGSGVPDFDTAEGFRTVLELVTQYQQLCIFWKVNYNFEDETVRKFLLSQLQKTRPVILDPAEPTGDVGGGDRWCWHLLAKEAKEWLSSPCFKDGTGNPIPPWKVPTMQTPGSCGARIHPIVNEMFSSRSHRILNNNSKRNF

SEQ ID NO: 24 - protein, 369 aa, synthetic construct
MAKNLSSTRIALCSTPAWRLDKFIEGHLLGDITFLTELRTDVNSISAFLKERCFQGAAHPMRVSRVVMGGSYNRYTVLKGRSEVDLLVFFNNLTCFDDQFKLQKEVIEEIQKHLCQFQQEKRLREKFKVQSSDQPNFRSVSFKLSYPKFQQEVEFHMQTAYDALYEVRRKENHNCEIYNKVYARLIRECTMLGKEGEFNICFMELQQDFLWKRPCELKNLICLVKHWYQLCKEKLREPLPPQYALELLTVYAWEHELPDKHETQTARGFRTVLELITKYLCLRIYWTLYYDVLHEQVNAYLYSQVKRVSPLILDPADPTWNVAGLNLQGWCILAEEAKAWLDYPCFKNRDGSRVSSWDVPPDKKGFVFL

SEQ ID NO: 25 - protein, 514 aa, synthetic construct
MALMQELYSTPASRLDSFVAQWLQPHREWKEEVLDAVRTVEEFLRQEHFQGKRGLDQDVRVLKVVKVGSFGNGTVLRSTREVELVAFLSCFHSFQEAAKHHKDVLRLIWKTMWQSQDLLDLGLEDLRMEQRVPDALVFTIQTRGTAEPITVTIVPAYRALGPSLPNSQPPPEVYVSLIKACGGPGNFCPSFSELQRNFVKHRPTKLKSLLRLVKHWYQQYVKARSPRANLPPLYALELLTIYAWEMGTEEDENFMLDEGFTTVMDLLLEYEVICIYWTKYYTLHNAIIEDCVRKQLKKERPIILDPADPTLNVAEGYRWDIVAQRASQCLKQDCCYDNRENPISSWNVKRARDIHLTVEQRGYPDFNLIVNPYEPIRKVKEKIRRTRGYSGLQRLSFQVPGSERQLLSSRCSLAKYGIFSHTHIYLLETIPSEIQVFVKNPDGGSYAYAINPNSFILGLKQQIEDQQGLPKKQQQLEFQGQVLQDWLGLGIYGIQDSDTLILSKKKGEALFPAS

SEQ ID NO: 26 - protein, 890 aa, Microbispora rosea
MPKYVYDFTEGNKDLKDLLGGKGANLAEMTNIGLPVPPGFTITAEACRHYLGHGGMPEGLEEEIGEHLAALEERMGKRLGQADDPLLVSVRSGAKFSMPGMMETVLNVGLNDDSVLGLAKQSGNDRFAWDSYRRLIQMFGKTVLDIDGELFEHAMDDLKGEREDTDLDAADLQRLVETFKGIVRERTGRDFPADPREQMDLAVKAVFDSWNAPRAILYRRQERIPADLGTAVNVVAMVFGNMGPDSGTGVAFTRDPGSGRQGVYGDYLRNAQGEDVVAGIRNTVPLQELETINPEAYRRLLDIMATLERHYRDLCDIEFTIERGRLWMLQTRVGKRTAAAAFCIATQLVDEGLIDMDEAVTRVTGDQLAQLMFPRFAATAGNHRLTTGMNASPGAAAGRAVFSSERAVELAGRGEAVILVRRETNPDDLAGMIAAHGVLTSRGGKTSHAAVVARGMGKTCVCGAEELEVDPHARRFTAPGGVVVDEGDVISIDGGTGAVYLGEVPVTPSPVAEYFEGEPAGDELVRAVDRIMTHADSVRRLAVRANADTPEDAARARRYGAQGIGLCRTEHMFLGERRRLVEDLILATTPEQRQAALDALEPLQTGDFTGIFEAMRGLPVTIRLIDPPLHEFLPDLTDLAVKVALADDRAANRADGRTGGGADDRDRRLLDAVKRLHEQNPMLGLRGVRLGLTVPGLFAMQVRAIAAAARRVEGACAEIMIPLVGAVQELEIVKEEAERILAEAGVEAAIGTMIEVPRAALTAGQIAEAAEFFSFGTNDLTQMTWGFSRDDVESAFFGTYLDLGVFGVSPFESVDREGVGRLMRIAVEEGRRTRPDLKLGICGEHGGDPDSVHFCHEIGLDYVSCSPFRIPVARLEAGRAALTCAQSDTR

SEQ ID NO: 27 - protein, 604 aa, Lactiplantibacillus plantarum
MKQTKQTKQTNILAGAAVIKVLEAWGVDHLYGIPGGSINSIMDALSAERDRIHYIQVRHEEVGAMAAAADAKLTGKIGVCFGSAGPGGTHLMNGLYDAREDHVPVLALIGQFGTTGMNMDTFQEMNENPIYADVADYNVTAVNAATLPHVIDEAIRRAYAHQGVAVVQIPVDLPWQQIPAEDWYASANSYQTPLLPEPDVQAVTRLTQTLLAAERPLIYYGIGARKAGKELEQLSKTLKIPLMSTYPAKGIVADRYPAYLGSANRVAQKPANEALAQADVVLFVGNNYPFAEVSKAFKNTRYFLQIDIDPAKLGKRHKTDIAVLADAQKTLAAILAQVSERESTPWWQANLANVKNWRAYLASLEDKQEGPLQAYQVLRAVNKIAEPDAIYSIDVGDINLNANRHLKLTPSNRHITSNLFATMGVGIPGAIAAKLNYPERQVFNLAGDGGASMTMQDLATQVQYHLPVINVVFTNCQYGFIKDEQEDTNQNDFIGVEFNDIDFSKIADGVHMQAFRVNKIEQLPDVFEQAKAIAQHEPVLIDAVITGDRPLPAEKLRLDSAMSSAADIEAFKQRYEAQDLQPLSTYLKQFGLDDLQHQIGQGGF

SEQ ID NO: 28 - protein, 591 aa, Streptococcus pneumoniae
MTQGKITASAAMLNVLKTWGVDTIYGIPSGTLSSLMDALAEDKDIRFLQVRHEETGALAAVMQAKFGGSIGVAVGSGGPGATHLINGVYDAAMDNTPFLAILGSRPVNELNMDAFQELNQNPMYNGIAVYNKRVAYAEQLPKVIDEACRAAVSKKGPAVVEIPVNFGFQEIDENSYYGSGSYERSFIAPALNEVEIDKAVEILNNAERPVIYAGFGGVKAGEVITELSRKIKAPIITTGKNFEAFEWNYEGLTGSAYRVGWKPANEVVFEADTVLFLGSNFPFAEVYEAFKNTEKFIQVDIDPYKLGKRHALDASILGDAGQAAKAILDKVNPVESTPWWRANVKNNQNWRDYMNKLEGKTEGELQLYQVYNAINKHADQDAIYSIDVGNTTQTSTRHLHMTPKNMWRTSPLFATMGIALPGGIAAKKDNPDRQVWNIMGDGAFNMCYPDVITNVQYDLPVINLVFSNAEYGFIKNKYEDTNKHLFGVDFTNADYAKIAEAQGAVGFTVDRIEDIDAVVAEAVKLNKEGKTVVIDARITQHRPLPVEVLELDPKLHSEEAIKAFKEKYEAEELVPFRLFLEEEGLQSRAIK

SEQ ID NO: 29 - protein, 309 aa, Amoracia rusticana
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 30 - protein, 741 aa, synthetic construct
MESRDHNNPQEGPTSSSGRRAAVEDNHLLIKAVQNEDVDLVQQLLEGGANVNFQEEEGGWTPLHNAVQMSREDIVELLLRHGADPVLRKKNGATPFILAAIAGSVKLLKLFLSKGADVNECDFYGFTAFMEAAVYGKVKALKFLYKRGANVNLRRKTKEDQERLRKGGATALMDAAEKGHVEVLKILLDEMGADVNACDNMGRNALIHALLSSDDSDVEAITHLLLDHGADVNVRGERGKTPLILAVEKKHLGLVQRLLEQEHIEINDTDSDGKTALLLAVELKLKKIAELLCKRGASTDCGDLVMTARRNYDHSLVKVLLSHGAKEDFHPPAEDWKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKINECVMKKMNKFYEKRGNFYQNTVGDLLKFIRNLGEHIDEEKHKKMKLKIGDPSLYFQKTFPDLVIYVYTKLQNTEYRKHFPQTHSPNKPQCDGAGGASGLASPGC

SEQ ID NO: 31 - protein, 652 aa, synthetic construct
MESRDHNNPQEGPTSSSGRRAAVEDNHLLIKAVQNEDVDLVQQLLEGGANVNFQEEEGGWTPLHNAVQMSREDIVELLLRHGADPVLRKKNGATPFILAAIAGSVKLLKLFLSKGADVNECDFYGFTAFMEAAVYGKVKALKFLYKRGANVNLRRKTKEDQERLRKGGATALMDAAEKGHVEVLKILLDEMGADVNACDNMGRNALIHALLSSDDSDVEAITHLLLDHGADVNVRGERGKTPLILAVEKKHLGLVQRLLEQEHIEINDTDSDGKTALLLAVELKLKKIAELLCKRGASTDCGDLVMTARRNYDHSLVKVLLSHGAKEDFHPPAEDWKPQSSHWGAALKDLHRIYRPMIGKLKFFIDEKYKIADTSEGGIYLGFYEKQEVAVKTFCEGSPRAQREVSCLQSSRENSHLVTFYGSESHRGHLFVCVTLCEQTLEACLDVHRGEDVENEEDEFARNVLSSIFKAVQELHLSCGYTHQDLQPQNILIDSKKAAHLADFDKSIKWAGDPQEVKRDLEDLGRLVLYVVKKGSISFEDLKAQSNEEVVQLSPDEETKDLIHRLFHPGEHVRDCLSDLLGHPFFWTWESRYRTLRNVGNESDIKTRKSESEILRLLQPGPSEHSKSFDKWTTKMSKLRHRQIIFPTTQNQ

SEQ ID NO: 32 - RNA, 36 nt, synthetic construct
  source 1..36: mol_type = other RNA
ttatcaaattcttatttgccccatttttttggttta

SEQ ID NO: 33 - protein, 592 aa, Danio rerio
MSSHRRPGCVSPTRPDEQPKAKARAKTSGKNEAQKKDKDVYVCESEAKLQETPSLKRGRSKKQKDDASKGDCKSLDNSESKSEEKHGDISKAGSVHKKTGENDRQDSAKDKLKSKRKTAPLDCESEGKNQQTANGKSGEKTALKRPSKKEKDHPNLDREATPENSESKLEQKPIASSSRNKSAPKKGFPGAKNACDASPAVSARSPDCKLKSTEKTAVKDALGDVLKATLDKLTIKKSERSKASRCVNEITEKVIAHLKQDTTWCADIERLRTGSYYENLKICEPDEFDVMLTVPVERVDIQNFDEAGAFYSIALKRHPNKHPLDKFLNEDKTIQASEMLSEFRDGVKKAVEKATDLPYKIKIQRKKPKCPAVTLEVTEGRKNISVDFVLGLKVHRASWPELTKDGFRVEHWLGRKVKSTMKRQPFYLVPKYEGIGNAEHDGVVARDSWRISFSHIEKEILKSHGHTKTCCEGREQKCCRKECLKLLKYLLQQLKNDESKSNKMSSFCSYHAKTTLLQACASRGTDIEWAYSELANCFQQLLEDFVKHLKNHQLPNFFIPSHNLLHHVSTSNCDFLAKEIEFQLNNTFPIFS

SEQ ID NO: 34 - protein, 592 aa, Xenopus tadpole
MKEMEEKSPKYIGIKRQTSSKINSCTTEAPNKSKASGITTGNSTASRKNSAKGEACKASVSKSGKATEGTSSNRTPDKAGNETAKTKLKDTKGKSKMKAPDNESAVSPMKATEGKEMDGAKDEASQKPVKAITKPSAGIKRNGASDKADKKPVGTVENAATCIQNNGALDKQDRTGEGNKALDKAYVEPEMTVRKLSTDGENKALQKADVEPLIKGKKAINMANKANAEPGSENDDKLTKHLNRTLKMQKLKMDEISKAAEQVKKLVKDLDKFIKSSKDPAFEGMEILNTGSYYEKVKISKPNEFDIMLKIPCGRLRISESDASGSFYTLTINRNPKHPLTNYVENGKISGQQMLDRLRELIKSKLSRLGTGINVERRNAASPAVTIKIGEISVDLVLALEVEGSWPSSTSEGMKIETWLGAKVKRDYKFEPMYFVAKQLKNTEEILWRISFSHIEKEILKNHGNAKTCCETEGVKCCRKQCLKLMKYLLEQLKSKGNRNMTHFCSYHAKTALLHSCTLYPKDDDWRPKDLAACFDRYIEDFMKCLKDARLPNFFIPSLNLFSEELIPKKCLDYLLRKLDKQKKLQYTLFDQ

SEQ ID NO: 35 - protein, 507 aa, Mus musculus
MEDPRRRTTAPRAKKPSAKRAPTQPSRTRAHAESCGPQRGARSRRAERDGDTTEKPRAPGPRVHPARATELTKDAQPSAMDAAGATARPAVRVPQQQAILDPELPAVREPQPPADPEARKVVRGPSHRRGARSTGQPRAPRGSRKEPDKLKKVLDKLRLKRKDISEAAETVNKVVERLLRRMQKRESEFKGVEQLNTGSYYEHVKISAPNEFDVMFKLEVPRIELQEYYETGAFYLVKFKRIPRGNPLSHFLEGEVLSATKMLSKFRKIIKEEVKEIKDIDVSVEKEKPGSPAVTLLIRNPEEISVDIILALESKGSWPISTKEGLPIQGWLGTKVRTNLRREPFYLVPKNAKDGNSFQGETWRLSFSHTEKYILNNHGIEKTCCESSGAKCCRKECLKLMKYLLEQLKKEFQELDAFCSYHVKTAIFHMWTQDPQDSQWDPRNLSSCFDKLLAFFLECLRTEKLDHYFIPKFNLFSQELIDRKSKEFLSKKIEYERNNGFPIFDKL

SEQ ID NO: 36 - protein, 376 aa, Rattus norvegicus
MEDPRRRTTAPRAKKPSAKRAPTRPSGTRATVSHAGSCGPQRGARSRRVERDGDTTEKPHTPAPRVRPRRAAERTEDAQPLATDAAGASERPAVREPQRQVILDPEPPAVPEPQAPEDPEAPTVARGPVRRRGARSIRQPRASQGSRKEPDKLKKVLDKLRLKRKEISAAAETVNKVVDQLLRRMQRRESEFKGVEQLNTGSYYEHVKISAPNEFDVMFKLEVPRIELEEYYETGAFYRVKFKRIPRGNPLSHFLEGEVLSATKVLSKFRELIKEEVKEIKDTDVTVEEEKPGSPAVTLLIRNPEEISVDIILALESKGSWPISTKGGLPIQDWLAVLSCTQECKRWKSFSRLVKCKDCREGCTRLVSVLCKDRKR

SEQ ID NO: 37 - protein, 522 aa, Homo sapiens
MQPWHGKAMQRASEAGATAPKASARNARGAPMDPTESPAAPEAALPKAGKFGPARKSGSRQKKSAPDTQERPPVRATGARAKKAPQRAQDTQPSDATSAPGAEGLEPPAAREPALSRAGSCRQRGARCSTKPRPPPGPWDVPSPGLPVSAPILVRRDAAPGASKLRAVLEKLKLSRDDISTAAGMVKGVVDHLLLRLKCDSAFRGVGLLNTGSYYEHVKISAPNEFDVMFKLEVPRIQLEEYSNTRAYYFVKFKRNPKENPLSQFLEGEILSASKMLSKFRKIIKEEINDIKDTDVIMKRKRGGSPAVTLLISEKISVDITLALESKSSWPASTQEGLRIQNWLSAKVRKQLRLKPFYLVPKHAKEGNGFQEETWRLSFSHIEKEILNNHGKSKTCCENKEEKCCRKDCLKLMKYLLEQLKERFKDKKHLDKFSSYHVKTAFFHVCTQNPQDSQWDRKDLGLCFDNCVTYFLQCLRTEKLENYFIPEFNLFSSNLIDKRSKEFLTKQIEYERNNEFPVFDEF

SEQ ID NO: 38 - protein, 544 aa, Phyllostomus discolor
MDPQHRRATRSASKAAASTLTVSGQITQGTPTKSAQTKSAPTKAAPPKPAGRKATRTNLSECPVVPDAAQPEEQMCCPTRASAPGRKKSAPGPQERPHVRARGARTQKAPLSAEDFLDGQTILPEPPAAREPPLPRVGPGGERGACSAREQKSQPWPMEDPGQHLLVPAPNLAGREAASCSWNLRAVLDKLKLRRYEISAAAEVVNKLVDHLLRRLQSSQSEFKLVEKLCTGSYYEQVKVSSPNEFDIMFKLDVPRIELEEYRDSGAYYFVKFKRNPKGNPLSHFLENEVLSASKMLSKFREIIKKEIKNIEDLDVIMERKKRGCPAVTLLIRKPKEISVDIILALQVKSSWPASTLEGLCIQTWLGRKVRRDLRLQPFYLVPKHAKEEDHFQENTWRLSFSHIEKDILKNHGKCKTCCETDGVRCCRKDCLKLMKYLLEQLKKKHGNRKELKKFCSYHVKTAFFHACTYHPEDSQWQPGDLELCFDRCLEDFLKCLREESLINYFIPGHNLFSQDKVDKISREFLLKEIEYERNNGFPVFKES

SEQ ID NO: 39 - protein, 341 aa, Hydra vulgaris
MVDAMNQEWWKKASDVVNAFHKEKAAIPSNAQRAREDLKENIAFLRTYLEFNYNLCDKVVFSGSAYEDLNISGDNIEFDVMLIAQRSNFLYLTECNNGVCKIRSNSPFLNYPFDEDFNIDSEKYRSFFFGLIQKWSNMMMTHKKKSFTLVNHGVATQMNVNDEKGILWYQVDLVPCFEAKSSLISEEKFYCVPKPIPNQRLYWRLSYSIDETQIAKKLSNDAKKCIRIIKALFKLETNGLFTKFTSYHIKTSAFYLKERGNWPNEENLGRSIYDFLVFIKESLKNGELKHFFDRTINLLDKIEVSTEQLANTINGWLKNEQKFLTKFSSSIDANIKQLAIK

SEQ ID NO: 40 - protein, 520 aa, Microplitis demolitor
MNGDEQNKKKLRSDAMFGKINQFVTLQKDETAKYNSLFTKVTESIVSILKEKDEVFKKYYRNTMYAGSFYKQTRVGQPKEFDLNLILCLPDIDSVKIEKGRPGFAKIKFDERKISSVWTEHKVLNKWLDSEGYLDNGKLRGWFEGMVKKSLNPSEKNSNKFRIYSKDSSSPLCEAEIKKSGPAFTLIVNDGSMEFAVDLVPVLEFSQSPPLSNFEKLKEPWHLVPKPLKNGDVPNQNWRYCFYHYEKEMLKKNGKVKPIIRHIKKLRDTQNWSILASYYIETLFFHVLSKNDFDQNESQTRLLVYMLKELSKAFKSGLLNYFWDKTFNLFGELTEDQIVGVQNRLDKIIKEIEADPTTMTQYFLTKEEQKKLKEIEEKEKTQPKKEVNVDKTNGLSSTFPLIRETKSEKNKKIIQESENRETSKNEIKALKEMVLSLKAEFKDFKNSIKQKPIDQTPETEGTDEKEMKKLLLLLINEVKQLKLNVGRLETKIDQTNEQIKNIKSQSTFFDVMETGVPLLN

SEQ ID NO: 41 - protein, 353 aa, Nicrophorus vespilloides
MEYSWLENKLQEINRKCIQINNKKSNNDSLNMIIGKLIDVMKDTSVLFKTLYERIIFNGSYYNNLKISKADEYDLDLILVLPVALETVLMLTPEAGYVDVKIDISKIKKLAYYRDILTPLEKLCENGIFSSLKIHSWFEGLVQKSLNIINASKYFPQYKLDMYKSGPAVTLTVHTPFSSYDMDLVPCLKLDKSLYPKGYKKQNCNHIYAVSKPNKDKEWRLSFIEQELSILRDNGHVKPAIRLLKFLRDQRDHKLIKSYFIVTVAMWDLETTNFNGKSLSFAFMTLLESLYKHICERKIPFYWNPNLNLLNKCHRDYLFNLENQLLNILKKLRSSNSNPNVLNEVFNFDIQES

SEQ ID NO: 42 - protein, 396 aa, Aethina tumida
MNKYNYMENVLQHINANVISLRDDEIKGNNIILKEILNIIIDKLKTKNIMFRKMYTCIFFGGSYYDGLRVGHPNEFDLDLLLTLHNLTKPIITKANEPGYVFLKLGNINNFLNIDDFKMYKQLSNLINKNGYLDVRKVLSWFEGIVTSSLNDIKEGSIYNFQIKGNTYKGTIHKGGPAFTLKIKGPNGSNMDIDLVPCFRFTEEHWPQGFKKSTSQQKSFFIVPKPLPDSTKSHYWRLSFQEQERELINNKGRLKPALRLLKQMRDTLNHHRIASYYLKTVFLWQVEEIGVDQSHWNSSLSYVFVCALKRYKEFLDSDNLPYFWEKKNNLLSGLHEDTLKNIRGSILKVLTDIESNNKDPNAIVKYLLTPEEQKRIMNGGNPQQSANAENGSCLSM

SEQ ID NO: 43 - protein, 383 aa, Asbolus verrucosus
MENILQDINKNFISLSNDEIRRNNIILDSVVQTFVGKMKEKDPLFNLMYRRVFYGGSFYDGLRVGKPKEFDLDLLLTLPNFAQPVLTTSNIPGFVFLKLENLDAWMRQPEAQRVGASFKKLLDGRNYLSTANVLVWMEGLVHRAANDLPARGSKRLLTTPVGTFEVSIHKGGPAMTLHICDSEIEIDVDLVACFVFGSNKWPTNRFRSNPVNSKPEFFIVPKKPRAPEDQPIQRYWRLSFQEQERVLIDNKQYLKPTVKLLKQLRDNLGHNFIASYYIKTVILHAVDERDDSFWRRPLSHVFMTILKDFKTYIERKKIPYYWNSNNNLLSGIGERTLENMNNRLRNVIRDMETRPETMWEYLGIKYTMYSKDSKKQFKELSEV

SEQ ID NO: 44 - protein, 398 aa, Tribolium castaneum
MENILNDINKRFISLPEEDVRGNKQILESVLRTFVEQMKTQDPLFKALFRRVFYGGSFYDGLKVGKPEEFDLDILLHIPIYAQPVLNESNVPGFVWLKLNNLDGWLRQPEGRVYKDFRKKFLADNDFLDTGKTLRWMESLVQKTLNTLPWVNNATCELTNEFGTFHINWWKGGPAMTLGISHSSGEKIMDVDLVACFVFSGDKWPINGYRSNPFPSTKPEFFIVPKKPQGPVNPQGRYWSLSFQEQERVLIDNKNRLKPAVKLIKKLKEKTHPNIASYYIKTVFLHIIEQKDQSFWNKSLREVFMTTLREYNEFIADQSIPYYWCRKNNLIGHLAPITLNNISNRIGYIIKDIENNPENIAKHLLTKEEYTKYIQGEDVMAEALPALPASQTSSCVII

(19) United States
(12) Patent Application Publication
(10) Pub. No.: US 2026/0234702 A1
(43) Pub. Date: Aug. 13, 2026

(54) COMPOSITIONS, METHODS, KITS, AND SYSTEMS FOR DETECTING DOUBLE-STRANDED RNA

(71) Applicant: New England Biolabs, Inc., Ipswich, MA (US)

(72) Inventors: Daviel Cardenas, North Andover, MA (US); David White, Malden, MA (US); Joseph M. Whipple, Beverly, MA (US); Lizhi Liu, Beverly, MA (US); Ivan R. Correa, JR., Hamilton, MA (US)

(73) Assignee: New England Biolabs, Inc., Ipswich, MA (US)

(21) Appl. No.: 19/438,326

(22) Filed: Dec. 31, 2025

Related U.S. Application Data
(60) Provisional application No. 63/740,714, filed on Dec. 31, 2024.

(51) Int. Cl. C12Q 1/6834; C12Q 1/6816

(52) U.S. Cl. CPC C12Q 1/6834; C12Q 1/6816

(57) ABSTRACT

The present disclosure relates, according to some embodiments, to compositions, methods, kits, and systems for detecting double-stranded RNA that include, for example, contacting a pattern recognition receptor (e.g., an oligoadenylate synthase 1; an Av cGAS-like receptor) with an RNA comprising or potentially comprising double-stranded RNA to conditionally produce (e.g., if dsRNA is present) pyrophosphate and, optionally, an oligonucleotide.

### CROSS-REFERENCE TO RELATED APPLICATIONS

This application also claims priority to U.S. Provisional Application No. 63/740,714 filed Dec. 31, 2024. The contents of all of the above are hereby incorporated in their entirety by reference.

### SEQUENCE LISTING STATEMENT

This disclosure includes a Sequence Listing submitted electronically in .xml format under the file name “NEB-508-US_seqlist.xml” created on Dec. 31, 2025, and having a size of 57,408 bytes. This Sequence Listing is incorporated herein in its entirety by this reference.

### BACKGROUND

RNA is a dynamic, multifunctional molecule capable of a variety of biological roles ranging from protein translation to gene regulation. An RNA molecule may be described or defined by its primary structure (its linear sequence of ribonucleotides), secondary structure (its base-paired stems and single-stranded loops), and/or tertiary structure (higher order intramolecular interactions which shape the overall RNA fold). An RNA molecule's structure can have an important impact on the behavior and function of RNA molecules in vitro and in vivo. RNA-based vaccines and therapeutics have been the focus of considerable research efforts. However, a key limitation in the success of such vaccines and therapeutics is the fact that RNA synthesis products often comprise some amount of double-stranded RNA (dsRNA). dsRNA activates cellular sensors which recognize viral genomes and may be a principle immunostimulatory species present in mRNA therapeutic formulations. Accordingly, it is desirable to reduce or minimize the amount of dsRNA present in RNA-based vaccines and therapeutics. This result is hindered by the limited means available for detecting dsRNA.

### SUMMARY

Accordingly, needs have arisen for improved compositions, methods, and kits for detecting dsRNA in a composition comprising RNA (e.g., RNA-based vaccines and therapeutics).

The present disclosure relates, according to some embodiments, to compositions, methods, kits, and systems for detecting double-stranded RNA that include, for example, contacting a pattern recognition receptor (e.g., an oligoadenylate synthase 1; an Av cGAS-like receptor) with an RNA comprising or potentially comprising double-stranded RNA to conditionally produce (e.g., if dsRNA is present) an oligonucleotide and pyrophosphate. A method may include, in some embodiments, contacting a pattern recognition receptor (e.g., an inactive dsRNA detector molecule) and a dsRNA to produce an activated pattern recognition receptor (e.g., an activated dsRNA detector molecule), and contacting the activated pattern recognition receptor (e.g., an activated dsRNA detector molecule) and a dsRNA detection substrate to produce a dsRNA detection intermediate. A dsRNA detection intermediate may be detected directly or indirectly. For example, a method may further comprise contacting the dsRNA detection intermediate and a conversion detector to produce an activated conversion detector, and contacting the activated conversion detector and a detection precursor to produce a detectable marker (e.g., indicating the presence of the dsRNA). In some embodiments, a pattern recognition receptor may comprise an oligoadenylate synthase (e.g., OAS1). A dsRNA detection intermediate may comprise, in some embodiments, pyrophosphate and/or an oligonucleotide (e.g., 2′-5′oligo(A)). A dsRNA may be present, for example, in a population of RNA molecules (e.g., a heterogeneous population of RNA molecules comprising or potentially comprising (a) fully single-stranded RNA molecules, (b) molecules each comprising at least one single-stranded portion and at least one double-stranded portion, and/or (c) fully dsRNA molecules. A population of RNA molecules may be or comprise one or more therapeutic RNAs. A population of RNA molecules may be or comprise an RNA library. A population of RNA molecules may be or comprise one or more naturally-occurring RNAs and/or one or more artificial RNAs. A population of RNA molecules may be or comprise one or more in vitro transcription products. One or more RNA molecules in a population of RNA molecules may comprise one or more modified nucleotides. A population of RNA molecules may be a cell-free population of RNA molecules.

According to some embodiments, a method may comprise contacting an inactive oligoadenylate synthase 1 (OAS1) and a dsRNA (e.g., a dsRNA in a population of RNA molecules) to produce pyrophosphate and an oligonucleotide, and detecting the pyrophosphate or the oligonucleotide, wherein the detected pyrophosphate and/or the detected oligonucleotide indicate that dsRNA is present in the population of RNA. In some embodiments, the population of RNA molecules may be or comprise one or more therapeutic RNAs. A population of RNA molecules may be or comprise an RNA library. A population of RNA molecules may be or comprise one or more naturally-occurring RNAs and/or one or more artificial RNAs. A population of RNA molecules may be or comprise one or more in vitro transcription products. One or more RNA molecules in a population of RNA molecules may comprise one or more modified nucleotides. In some embodiments, detecting pyrophosphate may comprise contacting the pyrophosphate (e.g., contacting a composition comprising or potentially comprising pyrophosphate) with a chemical detection reagent that conditionally changes its absorption and/or fluorescence (e.g., increases or decreases the intensity and/or wavelength of its absorption or fluroescence) upon contact with or otherwise in the presence of pyrophosphate and detecting the change in absportion and/or fluorescence (e.g., PhosphoWork™ Fluorimetric Pyrophosphate Assay Kit 21611). A chemical detection reagent (e.g., pyrophosphate detection reagent), in some embodiments, may comprise one or more metallochromic dyes (e.g., pyridylazo dyes) that can bind metals to form dye-metal complexes. Example chemical detection reagents include 2-(5-bromo-2-pyridylazo)-5-[N-propyl-N-(3-sulfopropyl)amino]phenol (abbreviated as 5-Bromo-PAPS; e.g., as a disodium salt dihydrate); 2-(5-nitro-2-pyridylazo)-5-[N-propyl-N-(3-sulfopropyl)amino]phenol (abbreviated as 5-Nitro-PAPS; e.g., as a disodium salt dihydrate); 4-(2-pyridylazo) resorcinol (PAR); 2-(3,5-dibromo-2-pyridylazo)-5-(N-ethyl-N-sulfopropylamino)benzene (abbreviated as 3,5-Dibromo-PAESA); 2-(3,5-dibromo-2-pyridylazo)-5-[N-ethyl-N-(3-sulfopropyl)amino]phenol (abbreviated as 3,5-diBromo-PAPS or DiBrPAPS); and hydroxynaphthol blue (HNB). A method comprising detecting the pyrophosphate, according to some embodiments, may include contacting the pyrophosphate with a pyruvate phosphate dikinase under conditions that produce pyruvate and inorganic phosphate, contacting the produced pyruvate and inorganic phosphate with a pyruvate oxidase under conditions that permit formation of hydrogen peroxide, and contacting the formed hydrogen peroxide with a peroxidase under conditions that permit conversion of 10-Acetyl-3,7-dihydroxyphenoxazine to 7-hydroxy-3H-phenoxazin-3-one. In some embodiments, detecting the oligonucleotide may comprise contacting the oligonucleotide with an inactive RNase L under conditions that produce an active RNase L and contacting the active RNase L with a quenched single-stranded RNA reporter (e.g., 5′-FAM-UUAUCAAAUUCUUAUUUGCCCCAUUUUUUUGGUUUA-3′Black Hole Quencher; SEQ ID NO:32) under conditions that permit cleavage of the reporter to yield a cleaved single-stranded RNA reporter and detecting fluorescence.

The present disclosure further provides methods comprising contacting an inactive oligoadenylate synthase 1 (OAS1) and a dsRNA to produce pyrophosphate and an oligonucleotide; and detecting the pyrophosphate or the oligonucleotide, wherein the dsRNA is included in a population of RNA and the detected pyrophosphate and/or the detected oligonucleotide indicate that dsRNA is present in the population of RNA. In some embodiments, the population of RNA molecules may comprise one or more modified nucleotides (e.g., alone or within one or more of the RNA molecules), at least one therapeutic RNA molecule, and/or one or more IVT products. Detecting the pyrophosphate may comprise, in some embodiments, contacting the pyrophosphate with a chemical detection reagent, wherein the chemical detection reagent comprises a metallochromic dye that can bind metals to form dye-metal complexes. A chemical detection reagent may be selected from 5-Bromo-PAPS, 5-Nitro-PAPS), PAR, 3,5-Dibromo-PAESA, 3,5-diBromo-PAPS, and HNB. According to some embodiments, detecting the pyrophosphate may comprise contacting the pyrophosphate with a chemical detection reagent that conditionally changes its absorption and/or fluorescence (e.g., increases or decreases the intensity and/or wavelength of its absorption or fluroescence) upon contact with or otherwise in the presence of pyrophosphate and detecting fluorescence. In some embodiments, detecting the pyrophosphate may comprise contacting the pyrophosphate with a pyruvate phosphate dikinase under conditions that produce pyruvate and inorganic phosphate, contacting the produced pyruvate with a pyruvate oxidase under conditions that permit formation of hydrogen peroxide, and contacting the formed hydrogen peroxide with a peroxidase under conditions that permit conversion of 10-Acetyl-3,7-dihydroxyphenoxazine to 7-hydroxy-3H-phenoxazin-3-one. In some embodiments, detecting the oligonucleotide may comprise contacting the oligonucleotide with an RNase L under conditions that produce an active RNase L and contacting the active RNase L with a quenched single-stranded RNA reporter under conditions that permit cleavage of the reporter to yield a cleaved single-stranded RNA reporter.

According to some embodiments, a method may comprise (a) contacting a dsRNA detector molecule (e.g., oligoadenylate synthase or a cGLR, for example, Asbolus verrucousus cGLR (Av cGLR)) with an RNA comprising or potentially comprising double-stranded RNA (dsRNA) to produce, if dsRNA is present, pyrophosphate and optionally, an oligonucleotide; and (b) detecting the pyrophosphate or the oligonucleotide. Detecting the pyrophosphate may comprise contacting the pyrophosphate with a chemical detection reagent, wherein the chemical detection reagent comprises a metallochromic dye that can bind metals to form dye-metal complexes. Examples of chemical detection reagents include 5-Bromo-PAPS, 5-Nitro-PAPS), PAR, 3,5-Dibromo-PAESA, 3,5-diBromo-PAPS, and HNB. In some embodiments, a chemical detection reagent may conditionally fluoresce upon contact with or otherwise in the presence of pyrophosphate and detecting fluorescence. A method may include detecting the pyrophosphate by contacting the pyrophosphate with a pyruvate phosphate dikinase under conditions that produce pyruvate and inorganic phosphate, contacting the produced pyruvate with a pyruvate oxidase under conditions that permit formation of hydrogen peroxide, and contacting the formed hydrogen peroxide with a peroxidase under conditions that permit conversion of 10-Acetyl-3,7-dihydroxyphenoxazine to 7-hydroxy-3H-phenoxazin-3-one. In some embodiments, a method may include detecting the oligonucleotide by contacting the oligonucleotide with an RNase L under conditions that produce an active RNase L and contacting the active RNase L with a quenched single-stranded RNA reporter under conditions that permit cleavage of the reporter to yield a cleaved single-stranded RNA reporter. A dsRNA (e.g., a dsRNA to be contacted with a dsRNA detection molecule) may be included in a population of RNA molecules (e.g., a cell-free population of RNA molecules). A population of RNA molecules may comprise one or more fully single-stranded RNA molecules and/or one or more RNA molecules each comprising at least one single-stranded portion and at least one double-stranded portion. In some embodiments, a population of RNA molecules may comprise one or more therapeutic RNA and/or one or more vaccine molecules.

The present disclosure further comprises dsRNA detection kits. A kit may comprise, for example, a dsRNA detector, a chemical detection reagent; and (optionally) one or more other reagents. For example, a dsRNA detection kit may comprise a dsRNA detector (e.g., OAS1 and Av cGLR), a chemical detection reagent (e.g, 5-Bromo-PAPS, 5-Nitro-PAPS, PAR, 3,5-Dibromo-PAESA, 3,5-diBromo-PAPS, and HNB); and (optionally) a buffer. In some embodiments, a kit may comprise one or more dNTPs, one or more rNTPs, one or more polymerases, one or more kinases, one or more oxidases, one or more peroxidases, one or more RNase Ls, or combinations thereof.

### BRIEF DESCRIPTION OF THE FIGURES

FIG. 1 shows an example method for detecting double-stranded RNA (dsRNA). As illustrated, a method may include activating 1010 a dsRNA detector molecule by contacting an inactive dsRNA detector molecule and dsRNA to produce an activated dsRNA detector molecule, producing 1020 a dsRNA detection intermediate by contacting the activated dsRNA detector molecule and a dsRNA detection substrate to produce the dsRNA detection intermediate, activating 1030 a conversion detector by contacting the dsRNA detection intermediate and a conversion detector to produce an activated conversion detector, and producing 1040 a detectable marker by contacting the activated conversion detector and a detection precursor to produce the detectable marker. In some embodiments, activating 1030 a conversion detector may be omitted and, if omitted, producing 1040 a detectable marker may comprise contacting the dsRNA detection intermediate and a detection precursor to produce the detectable marker.

FIG. 2 shows an example method for detecting dsRNA. As illustrated, a method may comprise producing 2010 an activated oligoadenylate synthase 1 (OAS1; any of SEQ ID NOS: 1-25) by contacting a dsRNA with an inactive OAS1 (iOAS1) to produce the activated OAS1, producing 2020 pyrophosphate (PPᵢ) by contacting the activated OAS1 and ATP to produce 2′-5′ oligoadenylate (2′-5′ oligo(A)) and the PPᵢ, producing 2032 pyruvate and inorganic phosphate (Pi) by contacting a pyruvate phosphate dikinase (PPDK; SEQ ID NO: 2), AMP, phosphoenol pyruvate (PEP), and the PPᵢ to produce the pyruvate, Pᵢ, and ATP, producing 2034 hydrogen peroxide (H₂O₂) by contacting the pyruvate, oxygen (O₂), Pᵢ, and a pyruvate oxidase (POx; e.g., SEQ ID NO:27, SEQ ID NO:28) to produce the H₂O₂, acetyl phosphate, and carbon dioxide (CO₂), and producing 2040 a detectable marker by contacting the H₂O₂, a horse radish peroxidase (HRP), and an HRP substrate (e.g., 10-Acetyl-3,7-dihydroxyphenoxazine (ADHP or Amplex Red) to produce the detectable marker (e.g., 7-hydroxy-3H-phenoxazin-3-one or resorufin). In some embodiments, a method may include conditionally producing hydrogen peroxide 2030 in response to production of pyrophosphate (e.g., optionally comprising steps 2032 and 2034).

FIG. 3A shows an example dose response of an embodiment of the method illustrated in FIG. 2 with increasing amounts of PolyI:C, a commonly used analog of dsRNA. As the dosage increases from 0.098 ng to 12.5 ng (X-axis) there is a corresponding increase in absorbance at 570 nm (Y-axis). The linear dose response breaks above 12.5 ng, as a 25 ng sample generates a reduced response.

FIG. 3B shows a portion of FIG. 3A highlighting signals for low input amounts (0.098 to 1.56 ng) of PolyI:C.

FIG. 4A shows example absorbance values for each PolyI:C dose from the example in FIG. 3A at each 30-minute time point. Samples from 0 to 12.5 ng of PolyI:C have a strong linear relationship whereas the signal for the 25 ng dose does not.

FIG. 4B shows an example linear regression analysis of values shown in FIG. 4A ranging from 0 to 12.5 ng of PolyI:C. The R² value was calculated to be 0.9972.

FIG. 5A shows an example dose response of the enzymatic cascade from FIG. 2 reacting to increasing amounts of an 80 basepair double-stranded RNA (80 bp dsRNA). As the dosage increases from 0.2 ng to 25.6 ng there is a corresponding increase in absorbance at 570 nm (OD).

FIG. 5B shows an example linear regression analysis for the values shown in FIG. 5A ranging from 0 to 25.6 ng of 80 bp dsRNA. Each point represents the 570 nm OD value (Y-axis) at the 30-minute time point for the dosage indicated on the X-axis. Unlike the experiment shown with PolyI:C in FIG. 4A, the dose response with 80 bp dsRNA displays a linear response through the 25 ng dose level. The R2 value was calculated to be 0.9807.

FIG. 6 shows an example comparison of dsRNA quantification using an embodiment of the method illustrated in FIG. 2 with dsRNA inputs of differing lengths, including a 300 bp dsRNA containing modified nucleotides (N1-methylpseudouridine). A 30 bp dsRNA did not activate OAS1 under the conditions tested. A 50 bp dsRNA elicited a weak response. This may indicate that the threshold for detection is between 30 and 50 bp of dsRNA under the conditions tested. This threshold may be attributed to a limited ability of short duplex RNAs to activate OAS1, which (without limiting any embodiment to any specific mechanism of action) may be a function of a property of the enzyme itself or a measure of the ability of short RNAs to retain a duplex conformation. An 80 bp dsRNA stimulated the same response as PolyI:C. Consistent with the reported function of modified bases in reducing immunogenicity, the modified 300 bp dsRNA stimulates a weaker response than the 80 bp unmodified dsRNA.

FIG. 7 shows an example wherein a method to detect dsRNA may comprise 2 steps (OAS1 and an RNA are contacted to react for a defined time and then, after the reaction is quenched, the reaction products are combined with pyrophosphate detection reagents) or combined into a single step (all enzymes and reagents are mixed and reacted together). Whereas the 2-step reaction tested plateaued after 20 minutes (FIG. 3A), the one-step reaction tested exhibited greater time dependence. Samples containing the same amount of input dsRNA (PolyI:C) showed increasing absorbance levels over time ranging from 30 to 90 minutes. Consistent with the method described in FIG. 2, a 1-step reaction generates increasing signal over an extended reaction time as OAS1 generates 2′-5′ oligoadenylate and pyrophosphate in response to a dsRNA stimulus. In a 2-step reaction, all pyrophosphate has already been produced by OAS1 and is available for reacting in subsequent enzymatic steps.

FIG. 8A shows an example dsRNA quantification assay result measuring a sample of in vitro transcribed erythropoietin (EPO) mRNA produced with different RNA polymerases (wild-type T7 RNA Polymerase at 37° C. or a cold-active RNA polymerase at 25° C.) and with or without modified nucleotide incorporation (N1-methylpseudouridine). 1 μg of RNA was used as input for each sample and signal is represented as percent dsRNA using PolyI:C as a standard. RNA produced with the cold-active RNA polymerase shows a lower level of dsRNA compared with the RNA produced by T7 RNA Polymerase. For both polymerases, incorporation of N1-methylpseudouridine dramatically reduced detectable levels of dsRNA.

FIG. 8B shows an example dsRNA quantification assay result measuring a sample of in vitro transcribed FLuc mRNA produced with different RNA polymerases (wild-type T7 RNA Polymerase at 37° C. or a cold-active RNA polymerase at 25° C.) and with or without modified nucleotide incorporation (N1-methylpseudouridine). Like the result in FIG. 8A, the transcript made with cold-active RNA polymerase has a lower level of dsRNA and N1-methylpseudouridine reduces signal significantly for both inputs.

FIG. 8C shows an example dsRNA quantification assay result measuring a sample of in vitro transcribed eGFP mRNA produced with different RNA polymerases (wild-type T7 RNA Polymerase at 37° C. or a cold-active RNA polymerase at 25° C.) and with or without modified nucleotide incorporation (N1-methylpseudouridine). Like the result the transcript made with cold-active RNA polymerase has a lower level of dsRNA and N1-methylpseudouridine reduces signal significantly for both inputs.

FIGS. 9A and 9B show example results detecting dsRNA in a 2-step reaction, the first step comprising dsRNA detection with OAS1 in a 50 μL reaction (1×Al buffer and 0.9 mM ATP at 37° C. for 20 min and 80° C. for 10 min) to generate PPi. FIG. 9A shows results observed in an example protocol having a second step comprising detecting PPi enzymatically (R²=0.9945). FIG. 9B shows results observed in an example protocol having a second step comprising dye-based PPi detection with diBrPAPS (R²=0.9950).

FIG. 10 shows example results showing substrate specificity and linearity of detecting dsRNA with OAS1.

FIG. 11 shows example results showing substrate specificity and linearity of detecting dsRNA with OAS1.

FIG. 12A shows example results of dsRNA detected in populations of EGFP RNA, EPO RNA, CLUC RNA and FLUC RNA by using a dsRNA detector molecule disclosed herein.

FIG. 12B shows example results of dsRNA detected in populations of EGFP RNA, EPO RNA, CLUC RNA and FLUC RNA by using a dsRNA binding antibody in a dot-blot assay.

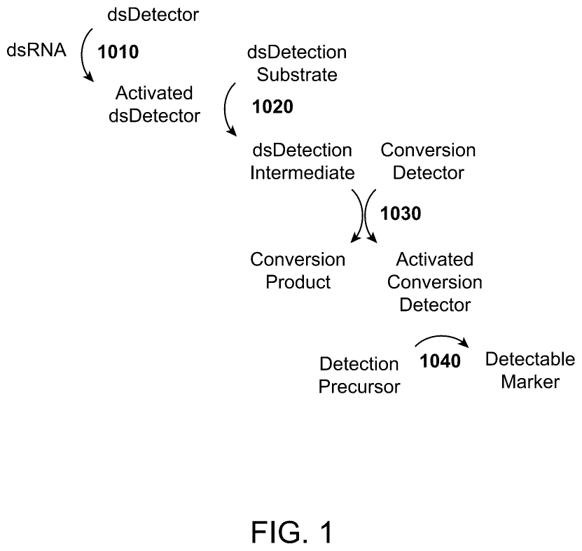

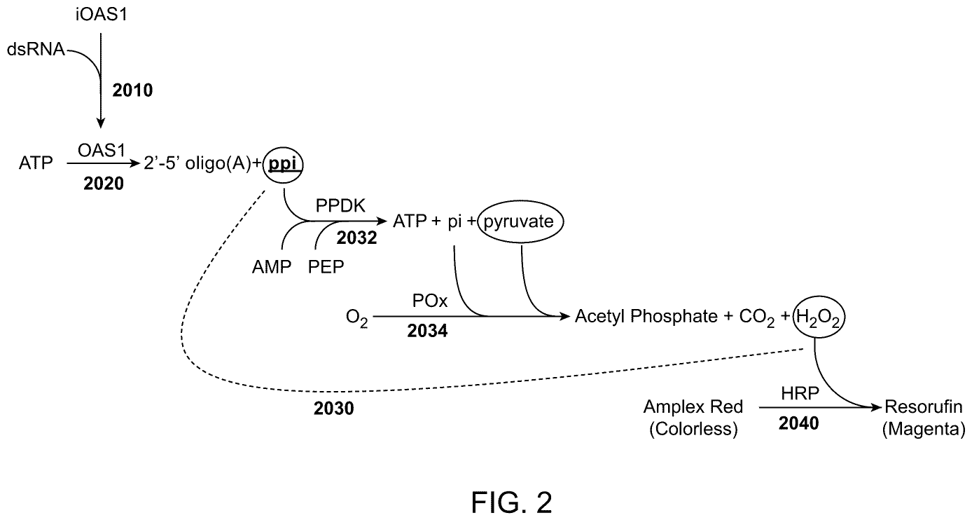

### BRIEF DESCRIPTION OF THE SEQUENCES

Some embodiments of this disclosure relate to the following provided sequences of example polynucleotides and/or example polypeptides.

SEQ ID NO: 1 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALGQLTGGYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLAESNSADDETDDPRRYQKYGYIGTHEYPHFSH
RPSTLQAASTPQAEEDWTCTIL

SEQ ID NO:2 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALGQLTGGYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLVRPPASSLPFIPAPLHEA

SEQ ID NO:3 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALGQLTGGYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLTQHTPGSIHPTGRRGLDLHHPLNASASWGKGL
QCYLDQFLHFQVGLLIQRGQSSSVSWCIIQDRTQVS

SEQ ID NO:4 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALGQLTGGYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLVNLTLVGRRNYTNN

SEQ ID NO:5 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALAHRGVHRPAERGRVLHLLHRTTERLPEAAPHQA
QEPHPPSQALVPKCMALPPGLCKKKLGKLPPQYALELLTVYAWERGSMKTHFNTAQGFRTVLE
LVINYQQLCIYWTKYYDFKNPIIEKYLRRQLTKPRPVILDPADPTGNLGGGDPKGWRQLAQEA
EAWLNYPCFKNWDGSPVSSWILLTQHTPGSIHPTGRRGLDLHHPLNASASWGKGLQCYLDQFL
HFQVGLLIQRGQSSSVSWCIIQDRTQVS

SEQ ID NO:6 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALAHRGVHRPAERGRVLHLLHRTTERLPEAAPHQA
QEPHPPSQALVPKCMALPPGLCKKKLGKLPPQYALELLTVYAWERGSMKTHFNTAQGFRTVLE
LVINYQQLCIYWTKYYDFKNPIIEKYLRRQLTKPRPVILDPADPTGNLGGGDPKGWRQLAQEA
EAWLNYPCFKNWDGSPVSSWILLAESNSADDETDDPRRYQKYGYIGTHEYPHFSHRPSTLQAA
STPQAEEDWTCTIL

SEQ ID NO:7 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALGQLTGGYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLMRQRLREVRSLAQGHQLTSGGNGIQAQWTLKP
VLMSLC

SEQ ID NO:8 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALAHRGVHRPAERGRVLHLLHRTTERLPEAAPHQA
QEPHPPSQALVPKCMALPPGLCKKKLGKLPPQYALELLTVYAWERGSMKTHFNTAQGFRTVLE
LVINYQQLCIYWTKYYDFKNPIIEKYLRRQLTKPRPVILDPADPTGNLGGGDPKGWRQLAQEA
EAWLNYPCFKNWDGSPVSSWILLMRQRLREVRSLAQGHQLTSGGNGIQAQWTLKPVLMSLC

SEQ ID NO:9 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALAHRGVHRPAERGRVLHLLHRTTERLPEAAPHQA
QEPHPPSQALVPKCMALPPGLCKKKLGKLPPQYALELLTVYAWERGSMKTHFNTAQGFRTVLE
LVINYQQLCIYWTKYYDFKNPIIEKYLRRQLTKPRPVILDPADPTGNLGGGDPKGWRQLAQEA
EAWLNYPCFKNWDGSPVSSWILLVRPPASSLPFIPAPLHEA

SEQ ID NO:10 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALAHRGVHRPAERGRVLHLLHRTTERLPEAAPHQA
QEPHPPSQALVPKCMALPPGLCKKKLGKLPPQYALELLTVYAWERGSMKTHFNTAQGFRTVLE
LVINYQQLCIYWTKYYDFKNPIIEKYLRRQLTKPRPVILDPADPTGNLGGGDPKGWRQLAQEA
EAWLNYPCFKNWDGSPVSSWILLVNLTLVGRRNYTNN

SEQ ID NO:11 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKCKK
KLGKLPPQYALELLTVYAWERGSMKTHFNTAQGFRTVLELVINYQQLCIYWTKYYDFKNPIIE
KYLRRQLTKPRPVILDPADPTGNLGGGDPKGWRQLAQEAEAWLNYPCFKNWDGSPVSSWILLT
QHTPGSIHPTGRRGLDLHHPLNASASWGKGLQCYLDQFLHFQVGLLIQRGQSSSVSWCIIQDR
TQVS

SEQ ID NO:12 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKCKK
KLGKLPPQYALELLTVYAWERGSMKTHFNTAQGFRTVLELVINYQQLCIYWTKYYDFKNPIIE
KYLRRQLTKPRPVILDPADPTGNLGGGDPKGWRQLAQEAEAWLNYPCFKNWDGSPVSSWILLA
ESNSADDETDDPRRYQKYGYIGTHEYPHFSHRPSTLQAASTPQAEEDWTCTIL

SEQ ID NO:13 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKCKK
KLGKLPPQYALELLTVYAWERGSMKTHENTAQGFRTVLELVINYQQLCIYWTKYYDFKNPIIE
KYLRRQLTKPRPVILDPADPTGNLGGGDPKGWRQLAQEAEAWLNYPCFKNWDGSPVSSWILLM
RQRLREVRSLAQGHQLTSGGNGIQAQWTLKPVLMSLC

SEQ ID NO:14 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKCKK
KLGKLPPQYALELLTVYAWERGSMKTHENTAQGFRTVLELVINYQQLCIYWTKYYDFKNPIIE
KYLRRQLTKPRPVILDPADPTGNLGGGDPKGWRQLAQEAEAWLNYPCFKNWDGSPVSSWILLV
RPPASSLPFIPAPLHEA

SEQ ID NO:15 is an example oligoadenylate synthase 1 amino acid sequence isoform from Homo sapiens.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALGQLTGSYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLVNLTLVGRRNYPIISEHAVNLQQTRRASLSYS
FQVA

SEQ ID NO:16 is an example oligoadenylate synthase 1 amino acid sequence.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALGQLTGSYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLVRPPASSLPFIPAPLHEAL

SEQ ID NO:17 is an example oligoadenylate synthase 1 amino acid sequence.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALGQLTGSYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILL

SEQ ID NO:18 is an example oligoadenylate synthase 1 amino acid sequence.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQSERAFSVKFEVQAPRW
GNPRALSFVLSSLQLGEGVEFDVLPAFDALGQLTGGYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLVRPPASSLPFIPAPLHEA

SEQ ID NO:19 is an example oligoadenylate synthase 1 amino acid sequence.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQRERAFSVKFEVQAPRW
GNPRALSFVLSSLQLGEGVEFDVLPAFDALGQLTGSYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLVRPPASSLPFIPAPLHEA

SEQ ID NO:20 is an example oligoadenylate synthase 1 amino acid sequence.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQSERAFSVKFEVQAPRW
GNPRALSFVLSSLQLGEGVEFDVLPAFDALGQLTGSYKPNPQIYVKLIEECTDLQKEGEFSTC
FTELQRDFLKQRPTKLKSLIRLVKHWYQNCKKKLGKLPPQYALELLTVYAWERGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLVRPPASSLPFIPAPLHEA

SEQ ID NO:21 is an example oligoadenylate synthase 1 amino acid sequence.

MMDLRNTPAKSLDKFIEDYLLPDTCFRMQINHAIDIICGFLKERCFRGSSYPVCVSKVVKGGS
SGKGTTLRGRSDADLVVFLSPLTTFQDQLNRRGEFIQEIRRQLEACQSERAFSVKFEVQAPRW
GNPRALSFVLSSLQLGEGVEFDVLPAFDALGQLTGSYKPNPQIYVKLIEECTDLQKEGEFSTC
FTELQRDFLKQRPTKLKSLIRLVKHWYQNCKKKLGKLPPQYALELLTVYAWEQGSMKTHFNTA
QGFRTVLELVINYQQLCIYWTKYYDFKNPIIEKYLRRQLTKPRPVILDPADPTGNLGGGDPKG
WRQLAQEAEAWLNYPCFKNWDGSPVSSWILLVRPPASSLPFIPAPLHEA

SEQ ID NO:22 is an example oligoadenylate synthase 1 amino acid sequence.

MGNGESQLSSVPAQKLGWFIQEYLKPYEECQTLIDEMVNTICDVLQEPEQFPLVQGVAIGGSY
GRKTVLRGNSDGTLVLFFSDLKQFQDQKRSQRDILDKTGDKLKFCLFTKWLKNNFEIQKSLDG
FTIQVFTKNQRISFEVLAAFNALSLNDNPSPWIYRELKRSLDKTNASPGEFAVCFTELQQKFF
DNRPGKLKDLILLIKHWHQQCQKKIKDLPSLSPYALELLTVYAWEQGCRKDNFDIAEGVRTVL
ELIKCQEKLCIYWMVNYNFEDETIRNILLHQLQSARPVILDPVDPTNNVSGDKICWQWLKKEA
QTWLTSPNLDNELPAPSWNVLPAPLFTTPGHLLDKFIKEFLQPNKCFLEQIDSAVNIIRTFLK
ENCFRQSTAKIQIVRGGSTAKGTALKTGSDADLVVFHNSLKSYTSQKNERHKIVKEIHEQLKA
FWREKEEELEVSFEPPKWKAPRVLSFSLKSKVLNESVSFDVLPAFNALGQLSSGSTPSPEVYA
GLIDLYKSSDLPGGEFSTCFTVLQRNFIRSRPTKLKDLIRLVKHWYKECERKLKPKGSLPPKY
ALELLTIYAWEQGSGVPDFDTAEGFRTVLELVTQYQQLCIFWKVNYNFEDETVRKFLLSQLQK
TRPVILDPAEPTGDVGGGDRWCWHLLAKEAKEWLSSPCFKDGTGNPIPPWKVPVKVI

SEQ ID NO:23 is an example oligoadenylate synthase 1 amino acid sequence.

MGNGESQLSSVPAQKLGWFIQEYLKPYEECQTLIDEMVNTICDVLQEPEQFPLVQGVAIGGSY
GRKTVLRGNSDGTLVLFFSDLKQFQDQKRSQRDILDKTGDKLKFCLFTKWLKNNFEIQKSLDG
FTIQVFTKNQRISFEVLAAFNALSLNDNPSPWIYRELKRSLDKTNASPGEFAVCFTELQQKFF
DNRPGKLKDLILLIKHWHQQCQKKIKDLPSLSPYALELLTVYAWEQGCRKDNFDIAEGVRTVL
ELIKCQEKLCIYWMVNYNFEDETIRNILLHQLQSARPVILDPVDPTNNVSGDKICWQWLKKEA
QTWLTSPNLDNELPAPSWNVLPAPLFTTPGHLLDKFIKEFLQPNKCFLEQIDSAVNIIRTFLK
ENCFRQSTAKIQIVRGGSTAKGTALKTGSDADLVVFHNSLKSYTSQKNERHKIVKEIHEQLKA
FWREKEEELEVSFEPPKWKAPRVLSFSLKSKVLNESVSFDVLPAFNALGQLSSGSTPSPEVYA
GLIDLYKSSDLPGGEFSTCFTVLQRNFIRSRPTKLKDLIRLVKHWYKECERKLKPKGSLPPKY
ALELLTIYAWEQGSGVPDFDTAEGFRTVLELVTQYQQLCIFWKVNYNFEDETVRKFLLSQLQK
TRPVILDPAEPTGDVGGGDRWCWHLLAKEAKEWLSSPCFKDGTGNPIPPWKVPTMQTPGSCGA
RIHPIVNEMESSRSHRILNNNSKRNF

SEQ ID NO:24 is an example oligoadenylate synthase 1 amino acid sequence.

MAKNLSSTRIALCSTPAWRLDKFIEGHLLGDITFLTELRTDVNSISAFLKERCFQGAAHPMRV
SRVVMGGSYNRYTVLKGRSEVDLLVFFNNLTCFDDQFKLQKEVIEEIQKHLCQFQQEKRLREK
FKVQSSDQPNFRSVSFKLSYPKFQQEVEFHMQTAYDALYEVRRKENHNCEIYNKVYARLIREC
TMLGKEGEFNICFMELQQDFLWKRPCELKNLICLVKHWYQLCKEKLREPLPPQYALELLTVYA
WEHELPDKHETQTARGFRTVLELITKYLCLRIYWTLYYDVLHEQVNAYLYSQVKRVSPLILDP
ADPTWNVAGLNLQGWCILAEEAKAWLDYPCFKNRDGSRVSSWDVPPDKKGFVFL

SEQ ID NO:25 is an example oligoadenylate synthase 1 amino acid sequence.

MALMQELYSTPASRLDSFVAQWLQPHREWKEEVLDAVRTVEEFLRQEHFQGKRGLDQDVRVLK
VVKVGSFGNGTVLRSTREVELVAFLSCFHSFQEAAKHHKDVLRLIWKTMWQSQDLLDLGLEDL
RMEQRVPDALVFTIQTRGTAEPITVTIVPAYRALGPSLPNSQPPPEVYVSLIKACGGPGNFCP
SFSELQRNFVKHRPTKLKSLLRLVKHWYQQYVKARSPRANLPPLYALELLTIYAWEMGTEEDE
NFMLDEGFTTVMDLLLEYEVICIYWTKYYTLHNAIIEDCVRKQLKKERPIILDPADPTLNVAE
GYRWDIVAQRASQCLKQDCCYDNRENPISSWNVKRARDIHLTVEQRGYPDFNLIVNPYEPIRK
VKEKIRRTRGYSGLQRLSFQVPGSERQLLSSRCSLAKYGIFSHTHIYLLETIPSEIQVFVKNP
DGGSYAYAINPNSFILGLKQQIEDQQGLPKKQQQLEFQGQVLQDWLGLGIYGIQDSDTLILSK
KKGEALFPAS

SEQ ID NO:26 is an example pyruvate phosphate dikinase amino acid sequence from Microbispora rosea.

MPKYVYDFTEGNKDLKDLLGGKGANLAEMTNIGLPVPPGFTITAEACRHYLGHGGMPEGLEEE
IGEHLAALEERMGKRLGQADDPLLVSVRSGAKFSMPGMMETVLNVGLNDDSVLGLAKQSGNDR
FAWDSYRRLIQMFGKTVLDIDGELFEHAMDDLKGEREDTDLDAADLQRLVETFKGIVRERTGR
DFPADPREQMDLAVKAVFDSWNAPRAILYRRQERIPADLGTAVNVVAMVFGNMGPDSGTGVAF
TRDPGSGRQGVYGDYLRNAQGEDVVAGIRNTVPLQELETINPEAYRRLLDIMATLERHYRDLC
DIEFTIERGRLWMLQTRVGKRTAAAAFCIATQLVDEGLIDMDEAVTRVTGDQLAQLMFPRFAA
TAGNHRLTTGMNASPGAAAGRAVFSSERAVELAGRGEAVILVRRETNPDDLAGMIAAHGVLTS
RGGKTSHAAVVARGMGKTCVCGAEELEVDPHARRFTAPGGVVVDEGDVISIDGGTGAVYLGEV
PVTPSPVAEYFEGEPAGDELVRAVDRIMTHADSVRRLAVRANADTPEDAARARRYGAQGIGLC
RTEHMFLGERRRLVEDLILATTPEQRQAALDALEPLQTGDFTGIFEAMRGLPVTIRLIDPPLH
EFLPDLTDLAVKVALADDRAANRADGRTGGGADDRDRRLLDAVKRLHEQNPMLGLRGVRLGLT
VPGLFAMQVRAIAAAARRVEGACAEIMIPLVGAVQELEIVKEEAERILAEAGVEAAIGTMIEV
PRAALTAGQIAEAAEFFSFGTNDLTQMTWGFSRDDVESAFFGTYLDLGVFGVSPFESVDREGV
GRLMRIAVEEGRRTRPDLKLGICGEHGGDPDSVHFCHEIGLDYVSCSPFRIPVARLEAGRAAL
TCAQSDTR

SEQ ID NO:27 is an example pyruvate oxidase amino acid sequence of Lactiplantibacillus plantarum.

MKQTKQTKQTNILAGAAVIKVLEAWGVDHLYGIPGGSINSIMDALSAERDRIHYIQVRHEEVG
AMAAAADAKLTGKIGVCFGSAGPGGTHLMNGLYDAREDHVPVLALIGQFGTTGMNMDTFQEMN
ENPIYADVADYNVTAVNAATLPHVIDEAIRRAYAHQGVAVVQIPVDLPWQQIPAEDWYASANS
YQTPLLPEPDVQAVTRLTQTLLAAERPLIYYGIGARKAGKELEQLSKTLKIPLMSTYPAKGIV
ADRYPAYLGSANRVAQKPANEALAQADVVLFVGNNYPFAEVSKAFKNTRYFLQIDIDPAKLGK
RHKTDIAVLADAQKTLAAILAQVSERESTPWWQANLANVKNWRAYLASLEDKQEGPLQAYQVL
RAVNKIAEPDAIYSIDVGDINLNANRHLKLTPSNRHITSNLFATMGVGIPGAIAAKLNYPERQ
VFNLAGDGGASMTMQDLATQVQYHLPVINVVFTNCQYGFIKDEQEDTNQNDFIGVEFNDIDFS
KIADGVHMQAFRVNKIEQLPDVFEQAKAIAQHEPVLIDAVITGDRPLPAEKLRLDSAMSSAAD
IEAFKQRYEAQDLQPLSTYLKQFGLDDLQHQIGQGGF

SEQ ID NO:28 is an example pyruvate oxidase amino acid sequence of Streptococcus pneumoniae.

MTQGKITASAAMLNVLKTWGVDTIYGIPSGTLSSLMDALAEDKDIRFLQVRHEETGALAAVMQ
AKFGGSIGVAVGSGGPGATHLINGVYDAAMDNTPFLAILGSRPVNELNMDAFQELNQNPMYNG
IAVYNKRVAYAEQLPKVIDEACRAAVSKKGPAVVEIPVNFGFQEIDENSYYGSGSYERSFIAP
ALNEVEIDKAVEILNNAERPVIYAGFGGVKAGEVITELSRKIKAPIITTGKNFEAFEWNYEGL
TGSAYRVGWKPANEVVFEADTVLFLGSNFPFAEVYEAFKNTEKFIQVDIDPYKLGKRHALDAS
ILGDAGQAAKAILDKVNPVESTPWWRANVKNNQNWRDYMNKLEGKTEGELQLYQVYNAINKHA
DQDAIYSIDVGNTTQTSTRHLHMTPKNMWRTSPLFATMGIALPGGIAAKKDNPDRQVWNIMGD
GAFNMCYPDVITNVQYDLPVINLVFSNAEYGFIKNKYEDTNKHLFGVDFTNADYAKIAEAQGA
VGFTVDRIEDIDAVVAEAVKLNKEGKTVVIDARITQHRPLPVEVLELDPKLHSEEAIKAFKEK
YEAEELVPERLFLEEEGLQSRAIK

SEQ ID NO:29 is an example horseradish root horseradish peroxidase amino acid sequence of Amoracia rusticana.

MQLTPTFYDNSCPNVSNIVRDTIVNELRSDPRIAASILRLHFHDCFVNGCDASILLDNTTSFR
TEKDAFGNANSARGFPVIDRMKAAVESACPRTVSCADLLTIAAQQSVTLAGGPSWRVPLGRRD
SLQAFLDLANANLPAPFFTLPQLKDSFRNVGLNRSSDLVALSGGHTFGKNQCRFIMDRLYNFS
NTGLPDPTLNTTYLQTLRGLCPLNGNLSALVDFDLRTPTIFDNKYYVNLEEQKGLIQSDQELF
SSPNATDTIPLVRSFANSTQTFFNAFVEAMDRMGNITPLTGTQGQIRLNCRVVNSNS

SEQ ID NO:30 is an example human RNase L amino acid sequence.

MESRDHNNPQEGPTSSSGRRAAVEDNHLLIKAVQNEDVDLVQQLLEGGA
NVNFQEEEGGWTPLHNAVQMSREDIVELLLRHGADPVLRKKNGATPFIL
AAIAGSVKLLKLFLSKGADVNECDFYGFTAFMEAAVYGKVKALKFLYKR
GANVNLRRKTKEDQERLRKGGATALMDAAEKGHVEVLKILLDEMGADVN
ACDNMGRNALIHALLSSDDSDVEAITHLLLDHGADVNVRGERGKTPLIL
AVEKKHLGLVQRLLEQEHIEINDTDSDGKTALLLAVELKLKKIAELLCK
RGASTDCGDLVMTARRNYDHSLVKVLLSHGAKEDFHPPAEDWKPQSSHW
GAALKDLHRIYRPMIGKLKFFIDEKYKIADTSEGGIYLGFYEKQEVAVK
TFCEGSPRAQREVSCLQSSRENSHLVTFYGSESHRGHLFVCVTLCEQTL
EACLDVHRGEDVENEEDEFARNVLSSIFKAVQELHLSCGYTHQDLQPQN
ILIDSKKAAHLADFDKSIKWAGDPQEVKRDLEDLGRLVLYVVKKGSISF
EDLKAQSNEEVVQLSPDEETKDLIHRLFHPGEHVRDCLSDLLGHPFFWT
WESRYRTLRNVGNESDIKTRKSESEILRLLQPGPSEHSKSFDKWTTKIN
ECVMKKMNKFYEKRGNFYQNTVGDLLKFIRNLGEHIDEEKHKKMKLKIG
DPSLYFQKTFPDLVIYVYTKLQNTEYRKHFPQTHSPNKPQCDGAGGASG
LASPGC

SEQ ID NO:31 is an example human RNase L amino acid sequence.

MESRDHNNPQEGPTSSSGRRAAVEDNHLLIKAVQNEDVDLVQQLLEGGA
NVNFQEEEGGWTPLHNAVQMSREDIVELLLRHGADPVLRKKNGATPFIL
AAIAGSVKLLKLFLSKGADVNECDFYGFTAFMEAAVYGKVKALKFLYKR
GANVNLRRKTKEDQERLRKGGATALMDAAEKGHVEVLKILLDEMGADVN
ACDNMGRNALIHALLSSDDSDVEAITHLLLDHGADVNVRGERGKTPLIL
AVEKKHLGLVQRLLEQEHIEINDTDSDGKTALLLAVELKLKKIAELLCK
RGASTDCGDLVMTARRNYDHSLVKVLLSHGAKEDFHPPAEDWKPQSSHW
GAALKDLHRIYRPMIGKLKFFIDEKYKIADTSEGGIYLGFYEKQEVAVK
TFCEGSPRAQREVSCLQSSRENSHLVTFYGSESHRGHLFVCVTLCEQTL
EACLDVHRGEDVENEEDEFARNVLSSIFKAVQELHLSCGYTHQDLQPQN
ILIDSKKAAHLADFDKSIKWAGDPQEVKRDLEDLGRLVLYVVKKGSISF
EDLKAQSNEEVVQLSPDEETKDLIHRLFHPGEHVRDCLSDLLGHPFFWT
WESRYRTLRNVGNESDIKTRKSESEILRLLQPGPSEHSKSFDKWTTKMS
KLRHRQIIFPTTQNQ

SEQ ID NO:32 is an example single-stranded RNA reporter sequence (U1 is 5′-FAM-U; A36 is A-3′Black Hole Quencher).

UUAUCAAAUUCUUAUUUGCCCCAUUUUUUUGGUUUA

SEQ ID NO:33 is an example of a Danio rerio (Zebra Fish) cGAS (“Dr-cGAS”).

MSSHRRPGCVSPTRPDEQPKAKARAKTSGKNEAQKKDKDVYVCESEAKL
QETPSLKRGRSKKQKDDASKGDCKSLDNSESKSEEKHGDISKAGSVHKK
TGENDRQDSAKDKLKSKRKTAPLDCESEGKNQQTANGKSGEKTALKRPS
KKEKDHPNLDREATPENSESKLEQKPIASSSRNKSAPKKGFPGAKNACD
ASPAVSARSPDCKLKSTEKTAVKDALGDVLKATLDKLTIKKSERSKASR
CVNEITEKVIAHLKQDTTWCADIERLRTGSYYENLKICEPDEFDVMLTV
PVERVDIQNFDEAGAFYSIALKRHPNKHPLDKFLNEDKTIQASEMLSEF
RDGVKKAVEKATDLPYKIKIQRKKPKCPAVTLEVTEGRKNISVDFVLGL
KVHRASWPELTKDGFRVEHWLGRKVKSTMKRQPFYLVPKYEGIGNAEHD
GVVARDSWRISFSHIEKEILKSHGHTKTCCEGREQKCCRKECLKLLKYL
LQQLKNDESKSNKMSSFCSYHAKTTLLQACASRGTDIEWAYSELANCFQ
QLLEDFVKHLKNHQLPNFFIPSHNLLHHVSTSNCDFLAKEIEFQLNNTF
PIFS

SEQ ID NO:34 is an example of a Xenopus tadpole (Frog) cGAS (“Xt-cGAS”).

MKEMEEKSPKYIGIKRQTSSKINSCTTEAPNKSKASGITTGNSTASRKN
SAKGEACKASVSKSGKATEGTSSNRTPDKAGNETAKTKLKDTKGKSKMK
APDNESAVSPMKATEGKEMDGAKDEASQKPVKAITKPSAGIKRNGASDK
ADKKPVGTVENAATCIQNNGALDKQDRTGEGNKALDKAYVEPEMTVRKL
STDGENKALQKADVEPLIKGKKAINMANKANAEPGSENDDKLTKHLNRT
LKMQKLKMDEISKAAEQVKKLVKDLDKFIKSSKDPAFEGMEILNTGSYY
EKVKISKPNEFDIMLKIPCGRLRISESDASGSFYTLTINRNPKHPLTNY
VENGKISGQQMLDRLRELIKSKLSRLGTGINVERRNAASPAVTIKIGEI
SVDLVLALEVEGSWPSSTSEGMKIETWLGAKVKRDYKFEPMYFVAKQLK
NTEEILWRISFSHIEKEILKNHGNAKTCCETEGVKCCRKQCLKLMKYLL
EQLKSKGNRNMTHFCSYHAKTALLHSCTLYPKDDDWRPKDLAACFDRYI
EDFMKCLKDARLPNFFIPSLNLFSEELIPKKCLDYLLRKLDKQKKLQYT
LFDQ

SEQ ID NO:35 is an example of a Mus musculus (Mouse) cGAS (“Mm-cGAS”).

MEDPRRRTTAPRAKKPSAKRAPTQPSRTRAHAESCGPQRGARSRRAERD
GDTTEKPRAPGPRVHPARATELTKDAQPSAMDAAGATARPAVRVPQQQA
ILDPELPAVREPQPPADPEARKVVRGPSHRRGARSTGQPRAPRGSRKEP
DKLKKVLDKLRLKRKDISEAAETVNKVVERLLRRMQKRESEFKGVEQLN
TGSYYEHVKISAPNEFDVMFKLEVPRIELQEYYETGAFYLVKFKRIPRG
NPLSHFLEGEVLSATKMLSKFRKIIKEEVKEIKDIDVSVEKEKPGSPAV
ILLIRNPEEISVDIILALESKGSWPISTKEGLPIQGWLGTKVRTNLRRE
PFYLVPKNAKDGNSFQGETWRLSFSHTEKYILNNHGIEKTCCESSGAKC
CRKECLKLMKYLLEQLKKEFQELDAFCSYHVKTAIFHMWTQDPQDSQWD
PRNLSSCFDKLLAFFLECLRTEKLDHYFIPKFNLFSQELIDRKSKEFLS
KKIEYERNNGFPIFDKL

SEQ ID NO:36 is an example of a Rattus norvegicus (Rat) cGAS (“Rn-cGAS”).

MEDPRRRTTAPRAKKPSAKRAPTRPSGTRATVSHAGSCGPQRGARSRRV
ERDGDTTEKPHTPAPRVRPRRAAERTEDAQPLATDAAGASERPAVREPQ
RQVILDPEPPAVPEPQAPEDPEAPTVARGPVRRRGARSIRQPRASQGSR
KEPDKLKKVLDKLRLKRKEISAAAETVNKVVDQLLRRMQRRESEFKGVE
QLNTGSYYEHVKISAPNEFDVMFKLEVPRIELEEYYETGAFYRVKFKRI
PRGNPLSHFLEGEVLSATKVLSKFRELIKEEVKEIKDTDVTVEEEKPGS
PAVILLIRNPEEISVDIILALESKGSWPISTKGGLPIQDWLAVLSCTQE
CKRWKSFSRLVKCKDCREGCTRLVSVLCKDRKR

SEQ ID NO:37 is an example of a Homo sapiens (Human) cGAS (“Hs-cGAS”).

MQPWHGKAMQRASEAGATAPKASARNARGAPMDPTESPAAPEAALPKAG
KFGPARKSGSRQKKSAPDTQERPPVRATGARAKKAPQRAQDTQPSDATS
APGAEGLEPPAAREPALSRAGSCRQRGARCSTKPRPPPGPWDVPSPGLP
VSAPILVRRDAAPGASKLRAVLEKLKLSRDDISTAAGMVKGVVDHLLLR
LKCDSAFRGVGLLNTGSYYEHVKISAPNEFDVMFKLEVPRIQLEEYSNT
RAYYFVKFKRNPKENPLSQFLEGEILSASKMLSKFRKIIKEEINDIKDT
DVIMKRKRGGSPAVTLLISEKISVDITLALESKSSWPASTQEGLRIQNW
LSAKVRKQLRLKPFYLVPKHAKEGNGFQEETWRLSFSHIEKEILNNHGK
SKTCCENKEEKCCRKDCLKLMKYLLEQLKERFKDKKHLDKFSSYHVKTA
FFHVCTQNPQDSQWDRKDLGLCFDNCVTYFLQCLRTEKLENYFIPEFNL
FSSNLIDKRSKEFLTKQIEYERNNEFPVFDEF

SEQ ID NO:38 is an example of a Phyllostomus discolor (Bat) cGAS (“Pd-cGAS”).

MDPQHRRATRSASKAAASTLTVSGQITQGTPTKSAQTKSAPTKAAPPKP
AGRKATRTNLSECPVVPDAAQPEEQMCCPTRASAPGRKKSAPGPQERPH
VRARGARTQKAPLSAEDFLDGQTILPEPPAAREPPLPRVGPGGERGACS
AREQKSQPWPMEDPGQHLLVPAPNLAGREAASCSWNLRAVLDKLKLRRY
EISAAAEVVNKLVDHLLRRLQSSQSEFKLVEKLCTGSYYEQVKVSSPNE
FDIMFKLDVPRIELEEYRDSGAYYFVKFKRNPKGNPLSHFLENEVLSAS
KMLSKFREIIKKEIKNIEDLDVIMERKKRGCPAVTLLIRKPKEISVDII
LALQVKSSWPASTLEGLCIQTWLGRKVRRDLRLQPFYLVPKHAKEEDHF
QENTWRLSFSHIEKDILKNHGKCKTCCETDGVRCCRKDCLKLMKYLLEQ
LKKKHGNRKELKKFCSYHVKTAFFHACTYHPEDSQWQPGDLELCFDRCL
EDFLKCLREESLINYFIPGHNLFSQDKVDKISREFLLKEIEYERNNGFP
VFKES

SEQ ID NO:39 is an example of a Hydra vulgaris (Hydra) cGLR (“Hv-cGLR”).

MVDAMNQEWWKKASDVVNAFHKEKAAIPSNAQRAREDLKENIAFLRTYL
EFNYNLCDKVVFSGSAYEDLNISGDNIEFDVMLIAQRSNFLYLTECNNG
VCKIRSNSPFLNYPFDEDFNIDSEKYRSFFFGLIQKWSNMMMTHKKKSF
TLVNHGVATQMNVNDEKGILWYQVDLVPCFEAKSSLISEEKFYCVPKPI
PNQRLYWRLSYSIDETQIAKKLSNDAKKCIRIIKALFKLETNGLFTKFT
SYHIKTSAFYLKERGNWPNEENLGRSIYDFLVFIKESLKNGELKHFFDR
TINLLDKIEVSTEQLANTINGWLKNEQKFLTKFSSSIDANIKQLAIK

SEQ ID NO:40 is an example of a Microplitis demolitor (Wasp) cGLR (“Md-cGLR”).

MNGDEQNKKKLRSDAMFGKINQFVTLQKDETAKYNSLFTKVTESIVSIL
KEKDEVFKKYYRNTMYAGSFYKQTRVGQPKEFDLNLILCLPDIDSVKIE
KGRPGFAKIKFDERKISSVWTEHKVLNKWLDSEGYLDNGKLRGWFEGMV
KKSLNPSEKNSNKFRIYSKDSSSPLCEAEIKKSGPAFTLIVNDGSMEFA
VDLVPVLEFSQSPPLSNFEKLKEPWHLVPKPLKNGDVPNQNWRYCFYHY
EKEMLKKNGKVKPIIRHIKKLRDTQNWSILASYYIETLFFHVLSKNDFD
QNESQTRLLVYMLKELSKAFKSGLLNYFWDKTFNLFGELTEDQIVGVQN
RLDKIIKEIEADPTTMTQYFLTKEEQKKLKEIEEKEKTQPKKEVNVDKT
NGLSSTFPLIRETKSEKNKKIIQESENRETSKNEIKALKEMVLSLKAEF
KDFKNSIKQKPIDQTPETEGTDEKEMKKLLLLLINEVKQLKLNVGRLET
KIDQTNEQIKNIKSQSTFFDVMETGVPLLN

SEQ ID NO:41 is an example of a Nicrophorus vespilloides (Beetle) cGLR (“Nv-cGLR”).

MEYSWLENKLQEINRKCIQINNKKSNNDSLNMIIGKLIDVMKDTSVLFK
TLYERIIFNGSYYNNLKISKADEYDLDLILVLPVALETVLMLTPEAGYV
DVKIDISKIKKLAYYRDILTPLEKLCENGIFSSLKIHSWFEGLVQKSLN
IINASKYFPQYKLDMYKSGPAVTLTVHTPFSSYDMDLVPCLKLDKSLYP
KGYKKQNCNHIYAVSKPNKDKEWRLSFIEQELSILRDNGHVKPAIRLLK
FLRDQRDHKLIKSYFIVTVAMWDLETTNFNGKSLSFAFMTLLESLYKHI
CERKIPFYWNPNLNLLNKCHRDYLFNLENQLLNILKKLRSSNSNPNVLN
EVFNFDIQES

SEQ ID NO:42 is an example of an Aethina tumida (Beetle) cGLR (“At-cGLR”).

MNKYNYMENVLQHINANVISLRDDEIKGNNIILKEILNIIIDKLKTKNI
MFRKMYTCIFFGGSYYDGLRVGHPNEFDLDLLLTLHNLTKPIITKANEP
GYVFLKLGNINNFLNIDDFKMYKQLSNLINKNGYLDVRKVLSWFEGIVT
SSLNDIKEGSIYNFQIKGNTYKGTIHKGGPAFTLKIKGPNGSNMDIDLV
PCFRFTEEHWPQGFKKSTSQQKSFFIVPKPLPDSTKSHYWRLSFQEQER
ELINNKGRLKPALRLLKQMRDTLNHHRIASYYLKTVFLWQVEEIGVDQS
HWNSSLSYVFVCALKRYKEFLDSDNLPYFWEKKNNLLSGLHEDTLKNIR
GSILKVLTDIESNNKDPNAIVKYLLTPEEQKRIMNGGNPQQSANAENGS
CLSM

SEQ ID NO:43 is an example of an Asbolus verrucosus (Beetle) cGLR (“Av-cGLR”).

MENILQDINKNFISLSNDEIRRNNIILDSVVQTFVGKMKEKDPLFNLMY
RRVFYGGSFYDGLRVGKPKEFDLDLLLTLPNFAQPVLTTSNIPGFVFLK
LENLDAWMRQPEAQRVGASFKKLLDGRNYLSTANVLVWMEGLVHRAAND
LPARGSKRLLTTPVGTFEVSIHKGGPAMTLHICDSEIEIDVDLVACFVF
GSNKWPTNRFRSNPVNSKPEFFIVPKKPRAPEDQPIQRYWRLSFQEQER
VLIDNKQYLKPTVKLLKQLRDNLGHNFIASYYIKTVILHAVDERDDSFW
RRPLSHVFMTILKDFKTYIERKKIPYYWNSNNNLLSGIGERTLENMNNR
LRNVIRDMETRPETMWEYLGIKYTMYSKDSKKQFKELSEV

SEQ ID NO:44 is an example of a Tribolium castaneum (Beetle) cGLR (“Tc-cGLR”).

MENILNDINKRFISLPEEDVRGNKQILESVLRTFVEQMKTQDPLFKALF
RRVFYGGSFYDGLKVGKPEEFDLDILLHIPIYAQPVLNESNVPGFVWLK
LNNLDGWLRQPEGRVYKDFRKKFLADNDFLDTGKTLRWMESLVQKTLNT
LPWVNNATCELTNEFGTFHINWWKGGPAMTLGISHSSGEKIMDVDLVAC
FVFSGDKWPINGYRSNPFPSTKPEFFIVPKKPQGPVNPQGRYWSLSFQE
QERVLIDNKNRLKPAVKLIKKLKEKTHPNIASYYIKTVFLHIIEQKDQS
FWNKSLREVFMTTLREYNEFIADQSIPYYWCRKNNLIGHLAPITLNNIS
NRIGYIIKDIENNPENIAKHLLTKEEYTKYIQGEDVMAEALPALPASQT
SSCVII

### DETAILED DESCRIPTION

Messenger RNA (mRNA) has rapidly become an important therapeutic modality for a wide spectrum of diseases including viral infection, microbial pathogens, cancers, and metabolic disorders. mRNA therapeutics may be manufactured by in vitro transcription (IVT) using phage-derived RNA polymerases, such as T7 RNA polymerase. However, IVT may produce undesired byproducts that may hinder the functionality or safety of the mRNA therapeutic. Double stranded RNA (dsRNA) is one such byproduct and its presence in a final drug product may induce unwanted innate immune responses, which can impact patient health and reduce therapeutic efficacy. In humans, dsRNA is recognized as a pathogen-associated molecular pattern, or PAMP, by multiple cellular pathways which may activate an undesirable innate immune response. Strategies to reduce dsRNA may include engineered or alternative RNA polymerases, modified nucleotide triphosphates, and post-transcriptional purification. However, to effectively employ strategies for dsRNA reduction, analytical tools to measure dsRNA throughout the mRNA manufacturing process are necessary. Current methods of detection often utilize antibodies, such as the J2 and K2 antibodies, to recognize dsRNA by immunoblot or Enzyme Linked Immunosorbent Assay (ELISA). These methods often provide only a qualitative measure of dsRNA presence, yield subjective results, have low reproducibility, and/or are not amenable to high-throughput analysis.

The present disclosure relates, in some embodiments, to compositions, methods, kits, and systems for dsRNA detection. Compositions may comprise, in some embodiments, an enzyme that produces pyrophosphate in the presence of dsRNA (e.g., upon or following contact with dsRNA). Example enzymes include OAS1, for example, human OAS1. Methods of detecting dsRNA, according to some embodiments, include contacting a sample possibly comprising dsRNA, an OAS1, and a nucleotide triphosphate (e.g., ATP) to produce pyrophosphate and an oligonucleotide (e.g., 2′-5′ oligo(A)), and detecting pyrophosphate and/or the oligonucleotide. According to some embodiments, a kit may include an OAS1 and a buffer. A kit may further include, for example, one or more chemical detection reagents and/or an NTP (e.g., ATP). A chemical detection reagent (e.g., pyrophosphate detection reagent), in some embodiments, may comprise metallochromic dyes (e.g., pyridylazo dyes) that can bind metals to form dye-metal complexes. Example chemical detection reagents include 2-(5-bromo-2-pyridylazo)-5-[N-propyl-N-(3-sulfopropyl)amino]phenol disodium salt dihydrate (abbreviated as 5-Bromo-PAPS), 2-(5-nitro-2-pyridylazo)-5-[N-propyl-N-(3-sulfopropyl)amino]phenol disodium salt dihydrate (abbreviated as 5-Nitro-PAPS), 4-(2-pyridylazo) resorcinol (PAR), 2-(3,5-dibromo-2-pyridylazo)-5-(N-ethyl-N-sulfopropylamino) benzene (3,5-Dibromo-PAESA), 3,5-diBromo-PAPS, HNB, and any of the dyes of U.S. Pat. Nos. 10,968,493, 11,512,342, 11,525,166, International Patent Publication No. WO2022040443, and International Patent Publication No. WO2023076772. A kit may further include, for example, one or more oligoadenylate detection reagents (e.g., RNase L). According to some embodiments, a system for detecting dsRNA may include a reactor means and a detector means.

### General Considerations

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Lists of example species within a particular genus may vary in length at different places throughout the disclosure. Species lists shortened for convenience shall not be construed to exclude example species listed elsewhere in the specification. Any recited method can be carried out in the order of events recited or in any other order which is logically possible.

Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified. Percent ranges with only one end point (e.g., ≥90% or ≤10%) optionally include a second endpoint at the maximum or minimum percentage (e.g., ≥90% includes a range of 90%-100% and ≤10% includes a range of 0%-10%). Ranges (including percent ranges) with only one end point (e.g., ≥90 or ≤10) optionally include a second endpoint 10% higher or 10% lower than the provided endpoint (e.g., ≥90 includes a range of 90-99 and ≤10 includes a range of 1-10). Concentration percentages are w/v percentages unless otherwise indicated.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

As used herein, “catalytically active” refers to the property of a molecule (e.g., a proteinaceous molecule or macromolecule) to function as a catalyst of one or more chemical reactions relative to one or more substrates and products. A catalytically active salt active nuclease or salt active nuclease variant, for example, hydrolyzes one or more polydeoxyribonucleic acids to yield (however briefly) products comprising at least one 5′-phosphorylated oligonucleotide. Catalytic activity of salt active nuclease and/or salt active nuclease variants may be assessed using existing techniques applied to one or more model substrates and/or one or more substrates of interest. For example, effective assays for catalytic activity of an OAS1 may include direct detection and quantification of 2′-5′ oligo(A) using chromatography or mass spectrometry and/or coupled enzymatic conversion of PPi to monophosphate (Pi) by a pyrophosphatase and colorimetric measurement of Pi. Catalytic activity may be assessed with respect to disappearance of substrate (e.g., ATP for OAS1; PPᵢ, AMP, phosphoenolpyruvate for PPDK; full-length RNA for RNase L) or appearance of product (e.g., PPᵢ, poly(A) for OAS1; ATP, Pᵢ, pyruvate for PPDK, cleaved RNA fragments for RNase L), and/or metrics that serve as a proxy thereof.

In the context of the present disclosure, “chemical detection reagent” refers to any protein, chemical, and/or other material that undergoes at least one conditional change in at least one detectable property upon contact with or otherwise in the presence of pyrophosphate. Detectable properties include light (e.g., infra red, visible, UV) reactivity (e.g., absorption, fluorescence), ionization, and solubility in a defined solvent among others. For example, a chemical detection reagent may conditionally changes its absorption and/or fluorescence (e.g., increases or decreases the intensity and/or wavelength of its absorption or fluroescence) upon contact with or otherwise in the presence of pyrophosphate. A chemical detection reagent (e.g., pyrophosphate detection reagent), in some embodiments, may comprise one or more metallochromic dyes (e.g., pyridylazo dyes) that can bind metals to form dye-metal complexes. Example chemical detection reagents include 2-(5-bromo-2-pyridylazo)-5-[N-propyl-N-(3-sulfopropyl)amino]phenol (abbreviated as 5-Bromo-PAPS; e.g., as a disodium salt dihydrate); 2-(5-nitro-2-pyridylazo)-5-[N-propyl-N-(3-sulfopropyl)amino]phenol (abbreviated as 5-Nitro-PAPS; e.g., as a disodium salt dihydrate); 4-(2-pyridylazo) resorcinol (PAR); 2-(3,5-dibromo-2-pyridylazo)-5-(N-ethyl-N-sulfopropylamino)benzene (abbreviated as 3,5-Dibromo-PAESA); 2-(3,5-dibromo-2-pyridylazo)-5-[N-ethyl-N-(3-sulfopropyl)amino]phenol (abbreviated as 3,5-diBromo-PAPS or DiBrPAPS); and hydroxynaphthol blue (HNB).

In the context of the present disclosure, “contact” refers to any physical, chemical, electrical, magnetic or other association between two materials (e.g., between two molecules). Contacting includes any process, method, workflow or other means of bringing two (or more) materials into contact with one another. Contacting, for example, an enzyme and a substrate or a binding protein and its corresponding target, may include providing suitable conditions (e.g., concentration, pH, solvent, buffer, space (volume), temperature, time) and other parameters for the two materials to associate (e.g., for an enzyme to operatively interact with its substrate or a binding protein to bind its target). Contacting may be achieved by any method that brings two (or more) materials into operative association with one another including mixing (e.g., in solution), pouring, pipetting, flowing, injecting, vortexing, transferring, incubating, emulsifying, agitating, spraying, adhering, or coating one material with, in to, or on to another.

In the context of the present disclosure, “container” refers to a human-made container including containers made using human-programed machines. A container may comprise one or more walls (e.g., defining an interior volume) and optionally one or more openings. Containers comprising one or more openings may further comprise one or more closures (e.g., removable closures) for some or all such openings. A closure optionally may comprise an aperture or a septum, for example, to provide fluid communication with a volume of the container and a connected or inserted tube or syringe. Examples of containers include boxes, cartons, bottles, tubes (e.g., test tubes, microcentrifuge tubes), plates (e.g., 96-well, 384-well plates), vials, pipette tips, and ampules. Containers and/or closures may comprise any desired material including paper, plastics, glass, silicone, composites, metals, alloys, or combinations thereof. Containers and/or closures may comprise materials that are compostable, recyclable, and/or sustainable.

In the context of the present disclosure and with respect to an amino acid residue or a nucleotide base position, “corresponding to” refers to positions that lie across from one another when sequences are aligned, e.g., by the BLAST algorithm. An amino acid position in a functional or structural motif in one polymerase may correspond to a position within a functionally equivalent functional or structural motif in another polymerase.

In the context of the present disclosure, “duplex” and “double stranded” refer to any conformation of a polynucleotide in which two polynucleotide strands (e.g., separate molecules or spatially separated portions of a single molecule) are arranged anti parallel to one another in a helix (e.g., A-form, B-form, Z-form) with complementary bases of each strand paired with one another (e.g., in Watson-Crick base pairs). Paired bases may be stacked relative to one another to permit pi electrons of the bases to be shared. A polynucleotide may have a homoduplex conformation (e.g., DNA:DNA or RNA:RNA) or a heteroduplex confirmation (e.g., DNA:RNA).

Duplex stability, in part, may be related to the ratio of complementary bases to mismatches (if any) in the two strands, ratio of pairs with three hydrogen bonds (e.g., G:C) to pairs with two hydrogen bonds (e.g., A:T, A:U) in the duplex, and the length of the strands with higher ratios and longer strands generally associated with higher stability. Duplex stability, in part, may be related to ambient conditions including, for example, temperature, pH, salinity, and/or the presence, concentration and identity of any buffer(s), denaturant(s) (e.g., formamide), crowding agent(s) (e.g., PEG), detergent(s) (e.g., SDS), surfactant(s), polysaccharide(s) (e.g., dextran sulfate), chelator(s) (e.g., EDTA), and nucleic acid(s) (e.g., salmon sperm DNA). A duplex polynucleotide may comprise one or more unpaired bases including, for example, a mismatched base, a hairpin loop, a single-stranded (5′ and/or 3′) end.

Duplex polynucleotides may have any desired length and/or any desired number of mismatched bases. For example, a duplex polynucleotide may have a length of ≤10 nucleotides, 10-25 nucleotides, 10-200 nucleotides, ≤50 nucleotides, 50-500 nucleotides, 50-2000 nucleotides, ≤2 kb, and/or 2-10 kb. Duplex polynucleotides may have any desired number of mismatched nucleotides, for example, over 90%, over 95%, over 97%, over 98 and/or over 99% sequence identity. Duplex polynucleotides (e.g., dsDNase substrates) may have no more than one mismatch per 10 nucleotides, no more than 1 mismatch per 100 nucleotides, no more than 2 mismatches per 100 nucleotides, and/or no more than 3 mismatches per 100 nucleotides.

In the context of the present disclosure, “expression system” refers to systems for producing a protein from a polynucleotide template comprising components to produce the protein according to an RNA template (e.g., enzymes, amino acids, an energy source), and (optionally) components to produce the RNA template according to another RNA template or a DNA template (e.g., enzymes, nucleotides, an energy source). An expression system may comprise a bacterial (e.g., Escherichia coli) or yeast (e.g., Kluyveromyces lactis or Pichia pastoris) expression system in which the protein is encoded by an RNA or DNA template within an expression cassette, a plasmid or other expression vector. An expression system may comprise a viral expression system in which the protein is encoded by an RNA or DNA template (e.g., in an expression cassette) within a viral genome or viral expression vector. Examples of cell-free expression systems may include or comprise cell extracts of Escherichia coli S30, rabbit reticulocytes or wheat germ, PUREXPRESS® (New England Biolabs, Ipswich, MA), an insect cell extract system (e.g., Promega #L1101), or HeLa cell lysate-based protein expression systems (e.g., Thermo Fisher Scientific #88882). An expression cassette may comprise, in some embodiments, an expression control sequence (e.g., promoter), a coding sequence encoding the gene product (e.g., protein) of interest (e.g., a vaccinia capping enzyme fusion), and/or one or more termination sequences (e.g., terminators). An expression control sequence (e.g., promoter) may comprise any promoter operative in a desired expression system, including, for example, a GAP promoter, an AOX1 promoter, a LAC4 promoter, a P350 hybrid promoter, a T7 promoter, a T5 promoter, a Ptac promoter, a Ptrc promoter, ParaBAD promoter, a PrhaBAD promoter, a Tet promoter or a PhoA phosphate-starvation promoter.

In the context of the present disclosure, “fusion” refers to two or more polypeptides, subunits, or proteins covalently joined to one another (e.g., by a peptide bond). For example, a protein fusion may refer to a non-naturally occurring polypeptide comprising a protein of interest covalently joined to a second polypeptide. Examples of a second polypeptide include a reporter protein (e.g., a green fluorescent protein), a purification tag (e.g., a 6×His or 8×His tag), and expression tag, a polynucleotide binding protein, an enzyme, a conjugation tag (e.g., a SNAP® tag), and a peptide linker (e.g., a flexible linker, an inflexible linker, a cleavable linker). Unless otherwise disclosed, the protein of interest may be nearer to the N-terminal end or nearer to the C-terminal end than the second polypeptide to which it is joined. A fusion protein may have one or more heterologous domains added to the N-terminus, C-terminus, and or the middle portion of the protein. A fusion may comprise a non-naturally occurring combined polypeptide chain comprising two proteins or two protein domains joined directly to each other by a peptide bond or joined through a peptide linker. If two parts of a fusion protein are “heterologous”, they are not part of the same protein in its natural state. In some embodiments, a fusion may comprise an OAS1 covalently joined to a second polypeptide. In some embodiments, an OAS1 may include a fusion to an exogenous DNA binding domain, examples of which are provided in Table 1 of U.S. Pat. No. 11,259,184. Other examples of fusion proteins include fusions of an OAS1 and a heterologous targeting sequence, a linker, an epitope tag, a detectable fusion partner, such as a fluorescent protein, β-galactosidase, luciferase and/or functionally similar peptides. Components of a fusion protein may be joined by one more peptide bonds, disulfide linkages, and/or other covalent bonds.

In the context of the present disclosure, “immobilized” refers to covalent attachment of an enzyme to a solid support with or without a linker. Examples of solid supports include beads (e.g., magnetic, agarose, polystyrene, polyacrylamide, chitin). Beads may include one or more surface modifications (e.g., 06-benzyleguanine, polyethylene glycol) that facilitate covalent attachment and/or activity of an enzyme of interest. For example, a support may comprise a ligand and an enzyme may have a receptor for such ligand or an enzyme may comprise a ligand and a support may comprise a receptor for such ligand. Receptor-ligand binding may be covalent or non-covalent. Non-covalent attachment (e.g., avidin:biotin, chitin:CBP) may be useful in some embodiments, for example, where the level of dissociation of the binding partner is deemed tolerable. A linker may be disposed between a support and an enzyme. For example, linker disposed between a support and an enzyme may have a first covalent bond to the support and a second covalent bond to the enzyme. An immobilized enzyme comprising a ligand-receptor attachment may have a linker disposed between the support and the ligand-receptor attachment, a linker disposed between the enzyme and the ligand-receptor attachment, or both. An immobilized enzyme comprising a linker may also comprise an optional covalent bond directly between the enzyme and the support. A linker may be of any desired length and have any desired range of motion. A peptide linker may comprise one or more repeats (e.g., 1-10 repeats) of glycine-serine.

In the context of the present disclosure, “in vitro transcription” (IVT) refers to a cell-free reaction in which a DNA template is copied by a DNA-directed RNA polymerase (e.g., an RNA polymerase) to produce a product that comprises one or more RNA molecules having a sequence copied from the template. For clarity, IVT optionally may include co-transcriptional capping.

In the context of the present disclosure, “modified nucleotide” refers to nucleotides having a modification on the sugars (e.g., 2′-fluororibose, ribose, 2′-deoxyribose, arabinose, and hexose); and/or in the phosphate groups (e.g., phosphorothioates and 5′-N-phosphoramidite linkages); and/or in the nucleotide base (e.g., as described in U.S. Pat. No. 8,383,340; WO 2013/151666; U.S. Pat. No. 9,428,535 B2; US 2016/0032316). Examples of modified nucleotides include pseudouridine and N1-methyl-pseudouridine.

In the context of the present disclosure, “non-naturally occurring” refers to a molecule (e.g., a polynucleotide, polypeptide, carbohydrate, or lipid) or composition that does not exist in nature. Such a molecule or composition may differ from naturally occurring molecules or compositions in one or more respects. For example, a polymer (e.g., a polynucleotide, polypeptide, or carbohydrate) may differ in the kind and arrangement of the component parts (e.g., nucleotide sequence, amino acid sequence, or sugar molecules). A polymer may differ from a naturally occurring polymer with respect to the molecule(s) to which it is linked. For example, a “non-naturally occurring” polypeptide (e.g., protein) may differ from naturally occurring polypeptides in its secondary, tertiary, or quaternary structure, by having (or lacking) a chemical bond (e.g., a covalent bond including a peptide bond, a phosphate bond, a disulfide bond, an ester bond, and ether bond, and others) to a lipid, a carbohydrate, a second polypeptide (e.g., a fusion protein), or any other molecule. Similarly, a “non-naturally occurring” polynucleotide or nucleic acid may comprise (or lack) one or more other modifications (e.g., an added label or other moiety) to the 5′-end, the 3′ end, and/or between the 5′- and 3′-ends (e.g., methylation) of the nucleic acid. A “non-naturally occurring” molecule or composition may differ from naturally occurring compositions in one or more of the following respects: (a) having components that are not combined in nature, (b) having components in ratios and/or concentrations not found in nature, (c) lacking one or more components otherwise found in naturally occurring molecules or compositions (e.g., a cell-free composition, a chromosome-free composition, a histone-free composition, a polymerase-free composition, a cell membrane-free composition), (d) having a form not found in nature (e.g., dried, freeze dried, lyophilized, crystalline, aqueous, immobilized), and (e) having one or more additional components beyond those found in nature (e.g., a buffering agent, a detergent, a dye, a solvent or a preservative).

In the context of the present disclosure, “oligoadenylate synthase” and “OAS1” refer to any naturally occurring enzyme or non-naturally occurring enzyme (e.g., an engineered enzyme) that converts adenosine triphosphate to oligo(A) (e.g., 2′-5′ oligo(A)) and pyrophosphate (PPᵢ) in the presence of and/or upon contact with double-stranded RNA. The double-stranded RNA may comprise a single molecule (e.g., folded back on itself) or two separate stands. A double-stranded RNA may consist of all duplex RNA with blunt ends and no mismatches. A double-stranded RNA may comprise at least one double-stranded portion (e.g., hairpins) and one or more single-stranded portions (e.g., overhanging ends, loops, bulges). An OAS1 may be catalytically active only when actually in contact with dsRNA or its catalytic activity may have a finite half-life after contact with dsRNA. The minimum length of uninterrupted duplex RNA necessary for a double-stranded RNA to activate an OAS1 may vary dependent on reaction conditions such as dsRNA concentration, modified nucleotide content, temperature, pH, salinity, divalent cation concentration, polyamine presence, and/or molecular crowding agents.

Catalytic activity of an OAS1 may persist across a range of salt concentrations, temperatures and/or pH. For example, an OAS1 may display catalytic activity under a range of conditions and/or following removal from exposure to such conditions. An OAS1 may have catalytic activity at and/or following exposure to a pH from X^{†} to Y^{†}, where X^{†} is any of pH 4, 4.5, 5, 5.5, 6, 6.5, 7, and Y^{†} is any of pH 6.5, 7, 7.5, 8, 8.5, 9, 9.5, 10, 10.5, 11 and X^{†}<Y^{†}.

Catalytic activity of an OAS1 may be denominated in units, wherein one unit is the amount of enzyme needed to convert ATP into oligo(A) and pyrophosphate in 20 minutes at 37° C. in a 50 μL reaction volume comprising 2 mM ATP and 1×NEBuffer r2.1. Specific activity of an OAS1 may be expressed in terms of units per milligram of OAS1 protein.

An OAS1 may comprise one or more amino acids in addition to any of SEQ ID NOS: 1-25. For example, an OAS1 may comprise (e.g., at its amino terminal end or carboxy terminal end) 1-25 amino acids. Such additional amino acids may enable, facilitate and/or enhance translation, expression, cellular sorting, inactivation (e.g., by including a protease recognition and/or cleavage site), and/or purification. Such additional amino acids may constitute a linker, for example, to a support (e.g., a magnetic bead) or another protein.

An OAS1 may comprise an amino acid sequence according to any of SEQ ID NOS: 1-25 and variants (e.g., ancestral reconstruction variants) thereof. For example, an OAS1 may have OAS1 activity and comprise an amino acid sequence having ≥30%, ≥35%, ≥40%, ≥45%, ≥50%, ≥55%, ≥60%, ≥65%, ≥70%, ≥75%, ≥80%, ≥85%, ≥90%, ≥92%, ≥93%, ≥95%, ≥96%, ≥97%, ≥98%, or ≥99% identity to any of SEQ ID NOS: 1-25. An OAS1 may comprise an RNA binding site and/or a catalytic site that have the same or substantially the same three dimensional structure as human OAS1. An OAS1 may be a human OAS1, a non-human mammalian OAS1, a plant OAS1, a yeast OAS1, a bacterial OAS1, a viral OAS1, or an artificial OAS1 (e.g., an ancestral reconstruction variant). A variant OAS1 may have an amino acid sequence sharing any desired degree of sequence identity with SEQ ID NOS: 1-25 up to (but excluding) 100% identity.

In the context of the present disclosure, “pattern recognition receptor” refers to a catalytically active molecule capable of recognizing a duplex polynucleotide (e.g., dsDNA, dsRNA, heteroduplex DNA:RNA). Examples of pattern recognition receptors may include (a) cyclic GMP-AMP synthase (cGAS), which senses dsDNA, (b) cGAS-like receptors (cGLRs), which may sense dsDNA, dsRNA, and/or heteroduplex DNA:RNA, and (c) OAS1, which may sense dsRNA. Examples of cGASs may include Danio rerio cGLR, Xenopus tadpoles cGLR, Mus musculus cGLR, Rattus norvegicus cGLR, Homo sapiens cGLR, and Phyllostomus discolor cGLR. Examples of cGLRs may include Hydra vulgaris cGLR, Microplitis demolitor cGLR, Nicrophorus vespilloides cGLR, Aethina tumida cGLR, Asbolus verrucosus cGLR (“Av cGLR”), and Tribolium castaneum cGLR. Examples of cyclic GMP-AMP synthases and cGLRs are described in Li et al., 2023, Cell 186:3261-3276.

With reference to an amino acid, “position” refers to the place such amino acid occupies in the primary sequence of a peptide or polypeptide numbered from its amino terminus to its carboxy terminus. A position in one primary sequence may correspond to a position in a second primary sequence, for example, where the two positions are opposite one another when the two primary sequences are aligned using an alignment algorithm (e.g., BLAST (Journal of Molecular Biology. 215 (3): 403-410) using default parameters (e.g., expect threshold 0.05, word size 3, max matches in a query range 0, matrix BLOSUM62, Gap existence 11 extension 1, and conditional compositional score matrix adjustment) or custom parameters). An amino acid position in one sequence may correspond to a position within a functionally equivalent motif or structural motif that can be identified within one or more other sequence(s) in a database by alignment of the motifs.

In the context of the present disclosure, an amino acid sequence having a percent identity to a reference sequence may be disclosed with or without specifying one or more of the variant positions. For example, if a 100-amino acid polypeptide is disclosed as having an amino acid sequence having at least 90% identity to a 100-amino acid reference sequence, the sequence may differ from the reference sequence at any positions up to 10. If a 100-amino acid polypeptide is disclosed as having an amino acid sequence having at least 90% identity to a 100-amino acid reference sequence and having a substitution at a given position, the sequence may differ from the reference sequence as noted at that position plus at any other positions up to 9.

In the context of the present disclosure, “pyrophosphate detection” refers to any available method for detection of inorganic pyrophosphate (PPᵢ). Methods may include, for example, enzymatically converting (e.g., using a PPDK) inorganic pyrophosphate to one or more products (e.g., ATP and/or inorganic phosphate), wherein the one or more products activate another enzyme (e.g., a pyruvate oxidase) to produce products including peroxide. Methods may include enzymatically detecting peroxide formation, for example, using a horseradish peroxidase to convert a non-fluorescent substrate to a fluorescent product (e.g., amplex red to resorufin. Kits for pyrophosphate detection include Abcam Pyrophosphate Assay Kit (Colorimetric/Fluorometric) ab234040, Cell Biolabs Pyrophosphate Assay Kit MET-5159, Invitrogen EnzChek™ Pyrophosphate Assay Kit E6645, PhosphoWork™ Fluorimetric Pyrophosphate Assay Kit 21611.

In the context of the present disclosure, “RNase L” refers to any naturally occurring nuclease or non-naturally occurring nuclease (e.g., an engineered nuclease) that becomes catalytically active in the presence of a dsRNA detection intermediate (e.g., 2′-5′ oligo(A) produced by an OAS1 contacting double-stranded RNA). Upon activation by a dsRNA detection intermediate, an RNase L cleaves a reporter (e.g., a quenched single stranded RNA reporter to generate a fluorescent signal). An RNase L may additionally require the presence of a cofactor such as a NTP (e.g., ATP). An RNase L may be catalytically active only when actually in contact with a dsRNA detection intermediate or its catalytic activity may have a finite half-life after contact with dsRNA detection intermediate. The minimum amount of dsRNA detection intermediate needed to activate an RNase L may vary dependent on reaction conditions such as dsRNA concentration, modified nucleotide content, temperature, pH, salinity, divalent cation concentration, polyamine presence, and/or molecular crowding agents.

Catalytic activity of an RNase L may persist across a range of salt concentrations, temperatures and/or pH. For example, an RNase L may display catalytic activity under a range of conditions and/or following removal from exposure to such conditions. An RNase L may have catalytic activity at and/or following exposure to a pH from X^{†} to Y^{†}, where X^{†} is any of pH 4, 4.5, 5, 5.5, 6, 6.5, 7, and Y^{†} is any of pH 6.5, 7, 7.5, 8, 8.5, 9, 9.5, 10, 10.5, 11 and X^{†}<Y^{†}.

Catalytic activity of an RNase L may be denominated in units, wherein one unit is the amount of enzyme needed to convert quenched single stranded RNA reporter into cleaved fluorescent RNA reporter in 20 minutes at 37° C. in a 50 μL reaction volume comprising 2 mM ATP, 25 nM 2′-5′ oligo(A), and 1×NEBuffer r2.1. Specific activity of an RNase L may be expressed in terms of units per milligram of RNase L protein.

An RNase L may comprise one or more amino acids in addition to any of SEQ ID NOS: 27-28. For example, an RNase L may comprise (e.g., at its amino terminal end or carboxy terminal end) 1-25 amino acids. Such additional amino acids may enable, facilitate and/or enhance translation, expression, cellular sorting, inactivation (e.g., by including a protease recognition and/or cleavage site), and/or purification. Such additional amino acids may constitute a linker, for example, to a support (e.g., a magnetic bead) or another protein.

An RNase L may comprise an amino acid sequence according to any of SEQ ID NOS: 27 and 28 and variants (e.g., ancestral reconstruction variants) thereof. For example, an RNase L may have RNase L activity and comprise an amino acid sequence having ≥30%, ≥35%, ≥40%, ≥45%, ≥50%, ≥55%, ≥60%, ≥65%, ≥70%, ≥75%, ≥80%, ≥85%, ≥90%, ≥92%, ≥93%, ≥95%, 96%, ≥97%, ≥98%, or ≥99% identity to any of SEQ ID NOS: 27-28. An RNase L may comprise an RNA binding site and/or a catalytic site that have the same or substantially the same three-dimensional structure as human RNase L. An RNase L may be a human RNase L, a non-human mammalian RNase L, a plant RNase L, a yeast RNase L, a bacterial RNase L, a viral RNase L, or an artificial RNase L (e.g., an ancestral reconstruction variant). A variant RNase L may have an amino acid sequence sharing any desired degree of sequence identity with SEQ ID NOS: 27 and 28 up to (but excluding) 100% identity.

In the context of the present disclosure, “substitution” refers to an amino acid residue at a position in a comparator amino acid sequence that differs with respect to a corresponding position of a reference amino acid sequence, where the comparator and reference sequences are at least 60% identical to each other or at least 70% identical to each other or at least 80% identical to each other. A reference sequence and comparator sequence may have the same length or similar lengths (e.g., differing by ≤12%, ≤5%, ≤1%). A substitute amino acid residue at a position, in addition to differing from the corresponding position of a reference amino acid sequence, may differ from the amino acid at the corresponding position of all naturally-occurring sequences that are at least 60% identical to each other or at least 70% identical to each other or at least 80% identical to the reference sequence. Optionally, a substitute amino acid may have different properties than the amino acid in the corresponding position of the reference sequence. Optionally, a substitute amino acid may have similar properties to the amino acid in the corresponding position of the reference sequence (a “conservative” substitution). For example, a non-polar amino acid (e.g., A, V, L, I, M, W, and F (and optionally C, G, and P) may substitute for another non-polar amino acid, a polar amino acid (e.g., N, Q, S, T, and Y) may substitute for another polar amino acid (e.g., C, D, E, H, K, N, P. Q, R, S, and T), a positively charged amino acid (H, K, and R) may substitute for another positively charged amino acid, and a negatively charged amino acid (e.g., D and E) may substitute for another negatively charged amino acid. A substitute amino acid may be a natural amino acid (e.g., replacing another natural amino acid or a non-natural amino acid). A substitute amino acid may be a non-natural amino acid (e.g., replacing a natural amino acid or another non-natural amino acid).

In the context of the present disclosure, “transcript” refers to a polynucleotide template for a polypeptide. A transcript may comprise RNA (e.g., ssRNA), a cap or cap analog, and/or a polyA tail. A transcript may be capable of translation in a cell (e.g., a bacterial cell and/or a yeast cell). For example, a transcript may be or comprise mRNA. A fusion transcript may comprise polynucleotide templates for two or more polypeptides in a single polynucleotide.

All publications, patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

### Compositions

The present disclosure related, in some embodiments, to compositions for detecting dsRNA. In some embodiments, a composition may comprise an inactive dsRNA detector molecule (e.g., an OAS1), which becomes active upon, during, and/or following contact with a molecule comprising dsRNA. An active dsRNA detector may have a catalytic activity that the corresponding inactive dsRNA detector does not.

According to some embodiments, a composition may be glycerol free, animal free, and/or endotoxin free. In this context, “free” refers to having a level of the subject material that is below a definable threshold (e.g., a detection threshold, a regulatory threshold) and/or a definable effect on another component of the composition or an intended reaction or reaction product.

Compositions, in some embodiments, may exclude whole cells and/or exclude cell extracts. For example, where a composition is configured to bind a target polynucleotide, it may be desirable or required to exclude cells and cell extracts that may interfere. Compositions may exclude, for example, enzymes or other materials that may digest or denature the dsRNA sought to be detected. Compositions may lack, according to some embodiments, one or more of operable cell membranes, ribosomes, nucleus, cytoplasm, mitochondria, and/or a cell wall.

Compositions, in some embodiments, may exclude whole cells and/or exclude cell extracts. For example, where a composition is configured to cleave a target polynucleotide, it may be desirable or required to exclude cells and cell extracts that may include unwanted enzymes or other materials that may nick a strand (e.g., nicking enzymes) or cleave (e.g., single-stranded and double-stranded nucleases) polynucleotides or that ligate (e.g., ligases) polynucleotides cut by Argonaute/guide complexes. Compositions may lack, according to some embodiments, one or more of operable cell membranes, ribosomes, nucleus, cytoplasm, mitochondria, and/or a cell wall.

### Kits

The present disclosure further relates to reagent kits for detecting dsRNA. For example, a kit may include a dsRNA detector (e.g., OAS1, Av cGLR), dNTPs, rNTPs, other enzymes (e.g., other polymerases, enzymes other than polymerases, or both), buffering agents, or combinations thereof. A kit comprising one or more other enzymes may comprise one or more enzymes that operate on catalytic products of the active dsRNA detector (e.g., PPDK, pyruvate oxidase, horse radish peroxidase, RNase L). Enzymes may be included in a storage buffer. Any suitable storage buffer may be used, for example, buffers comprising one or more of a cryoprotectant (e.g., a polyol such as glycerol, an antifreeze protein), a salt, a detergent, a reducing agent, a sugar, a chelator, and an antimicrobial agent and having a pH tolerated by the enzyme to be stored, for example, between pH 6 and 9. A composition or kit may include a reaction buffer which may be in concentrated form, and the buffer may contain additives (e.g. glycerol), salt (e.g. NaCl, KCl), reducing agent, EDTA or detergents, molecular crowding agents (e.g., polyethylene glycol (PEG), trehalose), among others. Detergents include nonionic detergents (e.g., t-octylphenoxypolyethoxyethanol), anionic detergents (e.g., alkylbenzene sulfonates), cationic detergents (e.g., alkylbenzene quaternary ammonium), and zwitterionic detergents. A composition or kit comprising dNTPs may include one, two, three of all four of dATP, dTTP, dGTP and dCTP. A kit comprising rNTPs may include one, two, three of all four of rATP, rUTP, rGTP and rCTP. A kit may further comprise one or more modified nucleotides. A kit may include a dsRNA, for example, a dsRNA control. A kit may optionally comprise one or more primers (random primers, bump primers, exonuclease-resistant primers, chemically-modified primers, custom sequence primers, or combinations thereof).

A reagent kit may be a non-natural collection of components configured, for example, for convenient storage, shipping, delivery, and/or use. A kit may include one or more containers, each comprising one or more materials and each combinable with one or more kit components or other materials to form one or more of the compositions disclosed herein and/or to perform one or more methods disclosed herein. One or more components of a kit may be included in one container for a single step reaction, or one or more components may be contained in one container, but separated from other components for sequential use or parallel use or controllable commencement of a desired condition or reaction. The contents of a kit may be formulated for use in a desired method or process. At least one container of a kit may exclude whole cells and/or exclude cell extracts.

A kit is provided that contains: (i) a dsRNA detector (e.g., OAS1); and (ii) a buffer. The dsRNA detector may have a lyophilized form or may be included in a buffer (e.g., a storage buffer or a reaction buffer in concentrated form). A kit may contain the dsRNA detector in a mastermix suitable for receiving and amplifying a template nucleic acid. A dsRNA detector may be a purified enzyme so as to contain substantially no DNA or RNA and no nucleases. The reaction buffer in (ii) and/or storage buffers containing the DNA polymerase in (i) may include non-ionic, ionic e.g. anionic or zwitterionic surfactants and crowding agents. A kit may include the dsRNA detector and the reaction buffer in a single tube or in different tubes.

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or subpackaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### Methods of Use

The present disclosure relates to methods of detecting dsRNA. A method may include contacting RNA (e.g., a dsRNA, a population of RNA comprising or potentially comprising dsRNA, an in vitro transcription product, a therapeutic RNA) and a dsRNA detector, wherein the dsRNA detector is rendered catalytically active by, upon, during, or following contact with dsRNA. An active dsRNA detector molecule may be contacted with a substrate to produce a product, which may be detected directly or indirectly. For example, a method may include contacting RNA and oligoadenylate synthase 1 (OAS1) (e.g., any of SEQ ID NOS: 1-25) to produce an active OAS1. Without limiting any particular embodiment to any particular mechanism of action, activating an OAS1 may include OAS1 binding a dsRNA and/or undergoing a conformational change. A conformational change may include and/or result in formation of a tetramer of OAS1 bound to the dsRNA or a duplex portion of an RNA having both duplex and single-stranded portions. A method may include contacting an active OAS1 and ATP to produce pyrophosphate and 2′-5′ oligoadenosine and optionally detecting (indirectly or directly) one or both products (the pyrophosphate and/or 2′-5′ oligoadenosine).

The present disclosure relates, in some embodiments, to methods of making RNA (e.g., a therapeutic RNA, a vaccine) by in vitro transcription and assaying one or more IVT products for the presence of dsRNA. For example, an IVT reaction may comprise contacting a template with an RNA polymerase to form a transcription product. A method may further comprise co-transcriptionally or post-transcriptionally capping the transcription product. A transcription product may be subject to a cleanup reaction, for example, to remove reaction by products (e.g., pyrophosphate) and/or one or more IVT enzymes. In some embodiments, a method may include detecting dsRNA in IVT products. For example, a method may comprise contacting an IVT product with a dsRNA detector molecule to produce a detection product. Methods may further include performing in vitro transcription in the presence of a dsRNA detector. For example, a method may include contacting in vitro transcription (in progress) and a dsRNA detector to form a detection product and detecting such detection product in real time (e.g., as the IVT reaction is proceeding; upon completion of the IVT reaction).

### Systems

The present disclosure relates, in some embodiments, to systems for detecting dsRNA. A system may include, according to some embodiments, a sample receiver, one or more reaction chambers, one or more reagent dispensers, and one or more detection means. For example, a sample receiver may include a dispenser, injection port or other portal for introducing a sample (e.g., liquid, powder or other solid) into a container (e.g., reaction chamber). One or more reagent dispensers may be configured to introduce a reagent (e.g., inactive OAS1, other enzymes, substrates) to a container (e.g., reaction chamber). In some embodiments, a system may include only one reaction chamber (sometimes referred to as one-pot). According to some embodiments, detection may be staged across two or more reaction chambers (e.g., in consecutive fluid communication with one another). For example, activation of OAS1 and production of 2′-5′ oligo(A) and pyrophosphate may be included in one container and products conveyed to a second reaction chamber. Each detection means may be configured to be in operable communication with a relevant reaction chamber. For example, if a method includes a horseradish peroxidase-mediated conversion of Amplex Red and H₂O₂ to Resorufin in a reaction chamber, a system may include an absorbance detector and/or a fluorescence detector configured to detect changes in the reaction chamber.

### EXAMPLES

Some specific example embodiments may be illustrated by one or more of the examples provided herein.

### Example 1: Materials

A plasmid encoding an example oligoadenylate synthase 1 designed and ordered from Genscript (Piscataway, NJ). PolyI:C and modified dsRNA standards were purchased from InvivoGen (San Diego, CA) and Areterna (Gaithersburg, MD), respectively. A Pyrophosphatase Assay Kit was purchased from Abcam and was used following the manufacturer's recommendations. More specifically, reactions were assayed for pyrophosphate production using a Pyrophosphate Assay Kit (Abcam, Cambridge, UK) following the manufacturer's instructions for a colorimetric assay. Unless otherwise specified all other reagents were provided by New England Biolabs, Inc. (NEB).

### Example 2: Determination of Linear Range of dsRNA Recognition by OAS1

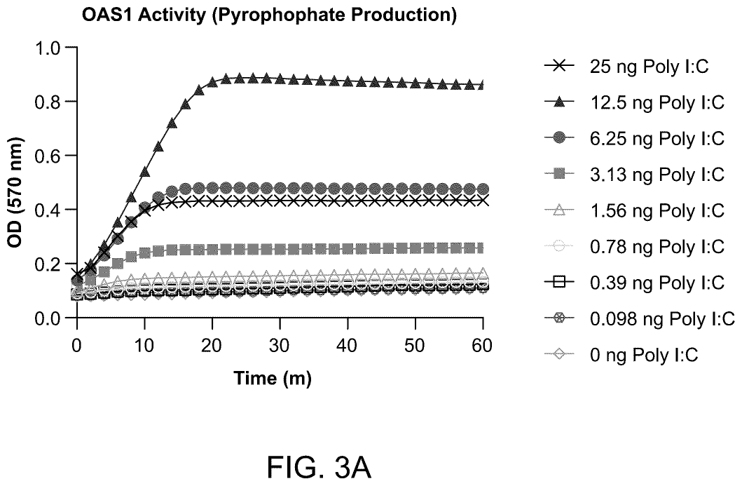

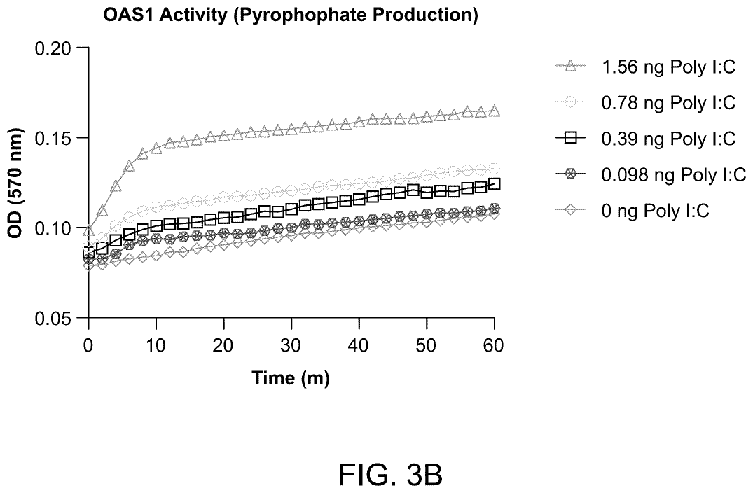

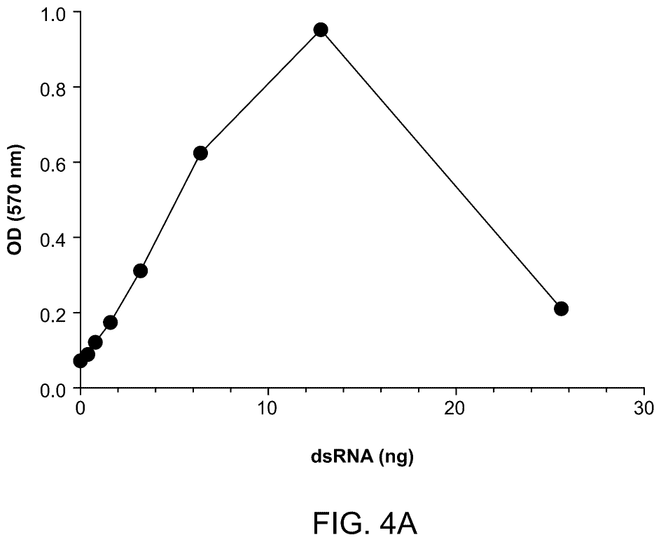

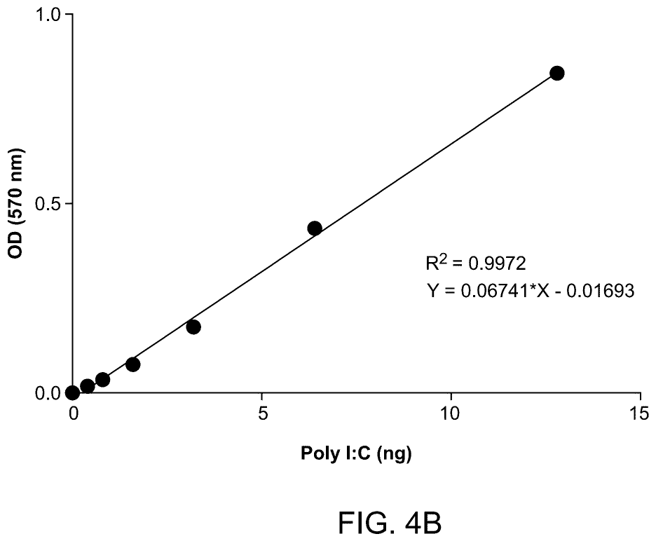

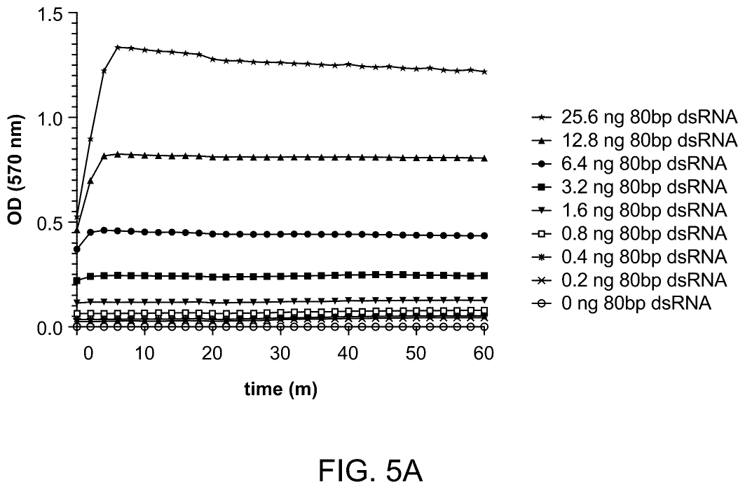

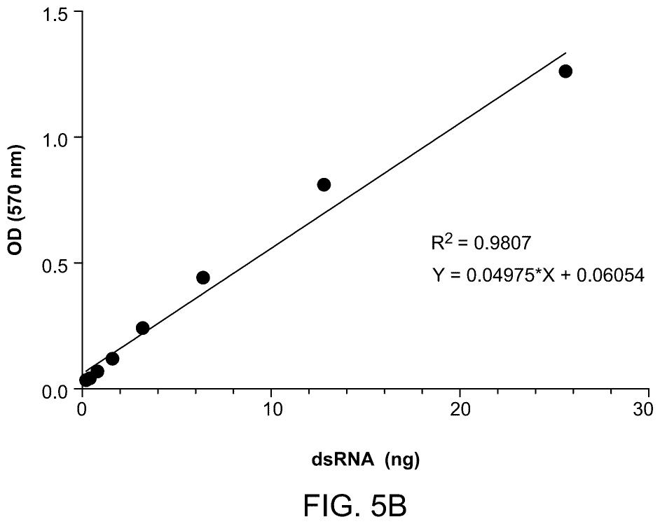

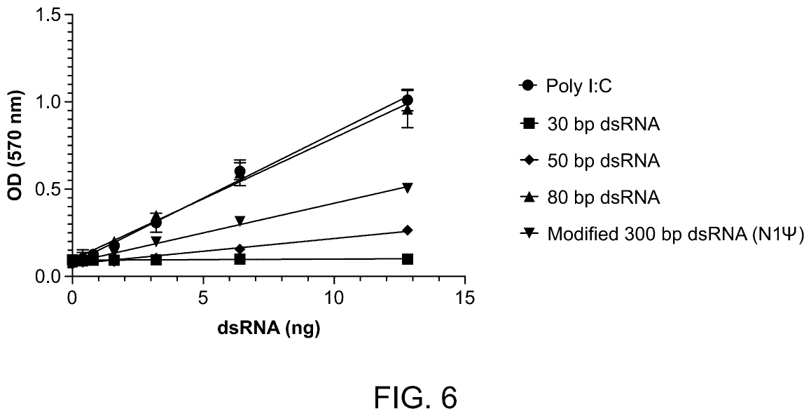

A serial dilution of dsRNA standards of different sizes and modifications was prepared and combined with 500 nM OAS1, 2 mM ATP, and 1×NEBuffer r2.1 in 50 μL reactions. The reactions were incubated at 37° C. for 20 minutes and quenched at 85° C. for 10 minutes. In a second step, the reactions were allowed to come to room temperature and were combined with 50 μL of the Kit manufacturer's pyrophosphate detection mix as per EXAMPLE 1. The final buffer composition of the combined reaction is 50% NEBuffer r2.1 and 50% of the recommended assay reaction buffer from EXAMPLE 1. The complete 100 μL reactions were transferred to a transparent 96-well plate and placed in a pre-warmed 37° C. SpectraMax M5 plate reader (Molecular Devices, San Jose, CA). Absorbance at 570 nm (OD (570 nm)) was measured continuously for 60 minutes. As shown in FIG. 3A, low molecular weight (LMW) poly I:C reacted with OAS1 displays a dose response from 0.4 ng to at least 12.8 ng of total dsRNA (FIG. 3A shows responses from to full set of samples, FIG. 3B shows a subset of only low input samples having 1.56 ng or less). Using GraphPad Prism (GraphPad Software, San Diego, CA), the absorbance values at the 30 minute time point were plotted against the concentration of dsRNA inputs, as shown in FIG. 4A. All samples, with the exception of 25 ng, demonstrate linear dose response as shown by linear regression analysis in FIG. 4B. This range of linear detection of dsRNA is within what is typically generated during IVT reactions (0.05-1% of total RNA or 0.5 to 10 ng of dsRNA per 1 μg of total RNA). As shown in FIG. 5A (OD 570 nm time course) and 5B (linear regression analysis of 30 minute time points), dsRNA detection reactions run using an 80 bp dsRNA (from NEB N0363) demonstrates very similar dose response parameters, though unlike PolyI:C linearity is maintained through 25.6 ng. Given the difference in performance using PolyI:C and 80 bp dsRNA inputs, several additional dsRNAs were tested, including 30 bp and 50 bp dsRNAs (both from NEB N0363) and a modified 300 bp dsRNA (m1Ψ) where uridine triphosphate was replaced with N1-methylpseudouridine triphosphate (m1ΨTP) (Areterna, Gaithersburg, MD). As shown in FIG. 6, a 30 bp dsRNA elicits no response while a 50 bp does, albeit relatively weakly as compared to 80 bp dsRNA. The modified 300 bp dsRNA was also detectable, though to a lesser degree than the 80 bp unmodified dsRNA. To determine if the individual dsRNA detection and pyrophosphate detection assays are compatible as a one-pot assay, we combined all reagents described above into 100 μL reactions and incubated them at 37° C. for 30, 60, or 90 minutes. OD (570 nm) was measured and a standard curve generated as described above. As shown in FIG. 7, the two reactions are compatible as a combined one-pot reaction. This is significant not only because it reduces handling steps, thus simplifying the reaction setup, but because it expands the linear range of dsRNA detection to at least 25.6 ng of dsRNA input.

### Example 3: Quantification of dsRNA Amounts in In Vitro Transcribed RNAs

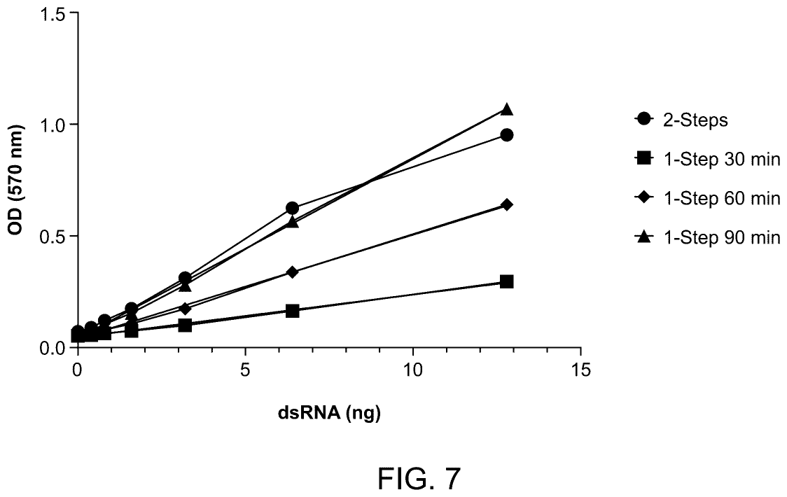

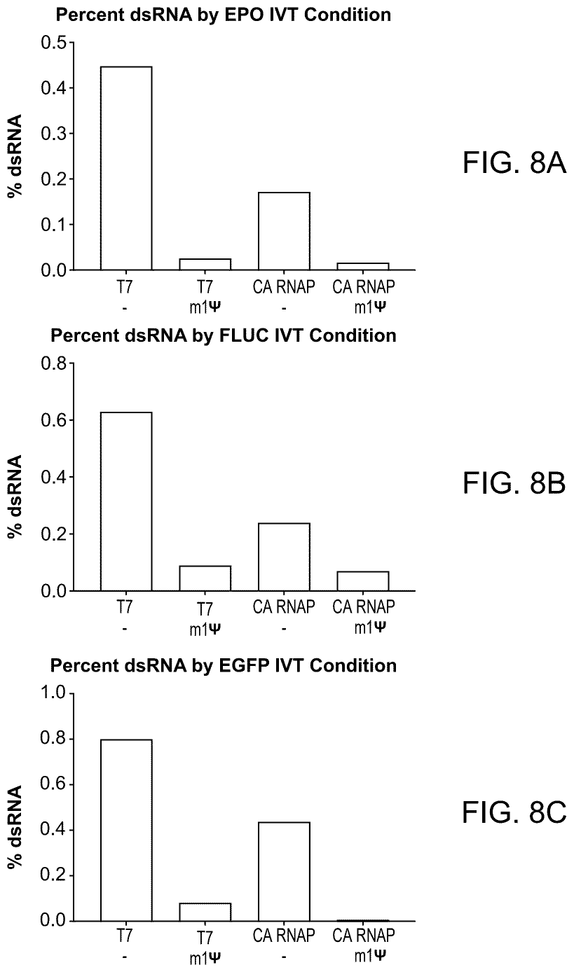

Firefly luciferase (FLUC), enhanced green fluorescent protein (EGFP), and erythropoietin (EPO) RNAs were prepared by IVT using 4 different IVT conditions (unmodified NTPS or m1ΨTP, using T7 RNA Polymerase or a cold-active RNA polymerase) and were purified using the Monarch Spin RNA Cleanup Kit (NEB, Inc.) to remove carryover pyrophosphate. The total RNA concentration was quantified using a NanoDrop 8000 spectrophotometer (Thermo Scientific, Waltham, MA). A total of 1 μg of IVT RNA was assayed using the two-pot dsRNA detection reaction detailed in EXAMPLE 2 and a standard curve was generated using LMW poly I:C. The background-subtracted absorbance values for all samples were utilized to compute the amount of dsRNA in the samples using the formula for the best fit line as described in EXAMPLE 2. For each sample, the absolute amount of dsRNA measured was divided by 1,000 ng of total RNA and multiplied by 100 to determine the percent dsRNA content. FIGS. 8A, B, and C illustrate that, for all 3 RNAs produced, the highest levels of dsRNA are detected using T7 RNA polymerase and standard NTPs. RNAs produced using cold-active RNA polymerase contain 2-3 fold less dsRNA. Incorporation of m1\′TP by either RNA polymerase dramatically reduces detectable dsRNA levels. Combined, these data demonstrate the functionality of the OAS1 and PPi detection assay as a suitable method for the quantification of dsRNA present in IVT RNAs prepared by several test conditions.

### Example 4: Comparison of PPi Detection by Enzyme Mix and diBrPAPS Dye

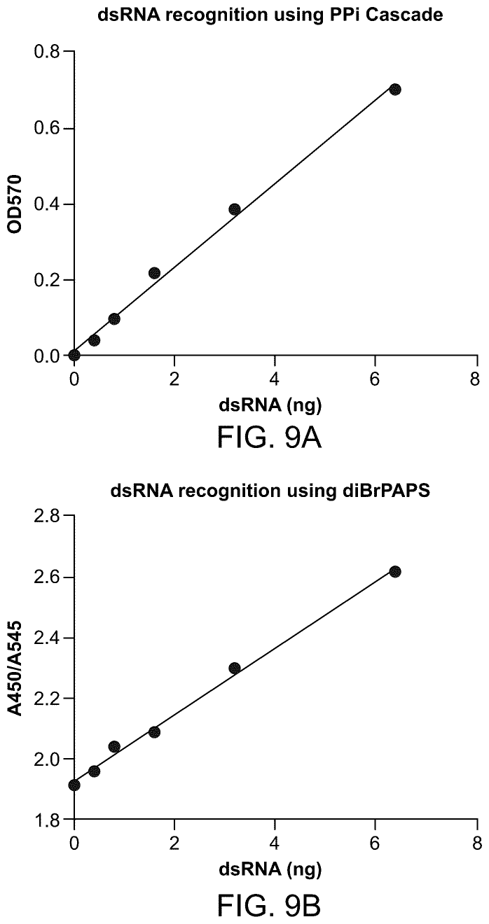

A 150 bp dsRNA standard was serially diluted from 6 ng and combined with 250 nM OAS1 and 0.9 mM ATP in 1×OAS1 buffer Al (10 mM Tris, pH 7; 50 mM NaCl; 4 mM MgCl₂; 100 mg/mL rAlb) for a final reaction volume of 50 μL. Reactions were incubated at 37° C. for 20 minutes, followed by heat inactivation at 80° C. for 10 minutes. The pyrophosphate produced by these reactions was then quantified using one of two methods, the first using the Kit manufacturer's detection mix described in EXAMPLE 1, and the second using a diBrPAPS dye mix. For the pyrophosphate detection mix, the final buffer consisted of 50% 1×Al reaction buffer and 50% of the Kit manufacturer's assay buffer recommended in EXAMPLE 1. The complete 100 μL reactions were transferred to a transparent 96-well plate and placed in a SpectraMax M5 plate reader (Molecular Devices, San Jose, CA) pre-warmed at 37° C. Absorbance at 570 nm was monitored continuously for 60 minutes. For the diBrPAPS method, 50 μL of OAS1 reaction was supplemented with 50 μL of dye mix containing 10 mM Tris, pH 9; 133 μM MnCl₂; 133 μM diBrPAPS; and 100 μM PPi. These mixtures were incubated at 80° C. for 10 minutes, allowed to equilibrate to room temperature, and transferred to a transparent 96-well plate. Finally, an end-point measurement of absorbance was measured at 450 nm and 545 nm on the SpectraMax M5 plate reader. The ratio between 450/545 changes depending on the amount of dsRNA present in the OAS1 reaction. As shown in FIGS. 9A and 9B, both methods produce comparable linear dose-responses to the 150 bp dsRNA on the 0.4 ng to 6 ng range.

### Example 5: Determination of Specificity of Substrate Recognition by Av cGLR

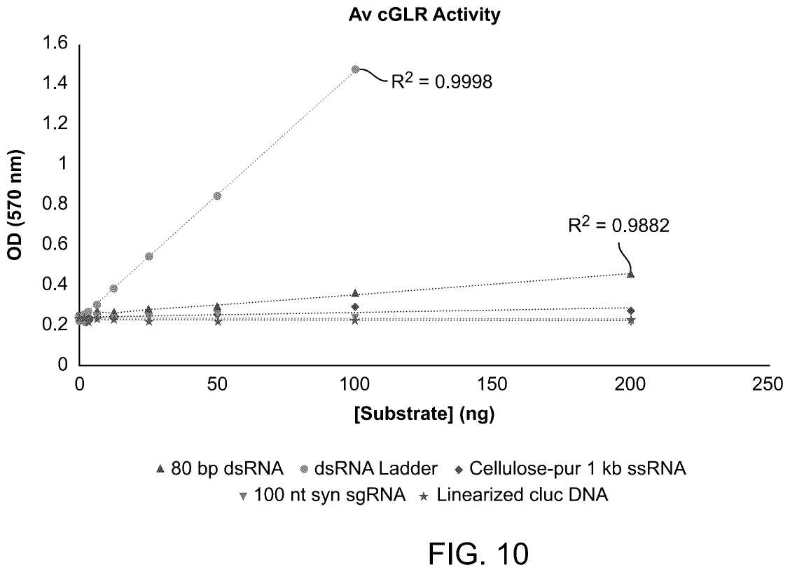

A serial dilution of several nucleic acid standards (from 1.56-200 ng) was prepared and combined with 300 nM Av cGLR, 1 mM ATP, 1 mM GTP, and 1×NEBuffer r2.1 in 50 μL reactions. The reactions were incubated at 37° C. for 75 minutes and quenched at 85° C. for 10 minutes. In a second step, the reactions were allowed to come to room temperature and were combined with 50 μL of the Kit manufacturer's pyrophosphate detection mix as per EXAMPLE 1. The final buffer composition of the combined reaction is 50% NEBuffer r2.1 and 50% of the Kit manufacturer's assay reaction buffer from EXAMPLE 1. The complete 100 μL reactions were transferred to a transparent 96-well plate and placed in a pre-warmed 37° C. SpectraMax M5 plate reader (Molecular Devices, San Jose, CA). Absorbance at 570 nm (OD (570 nm)) was measured continuously for 40 minutes. Using GraphPad Prism (GraphPad Software, San Diego, CA), the absorbance values at the 30 minute time point were plotted against the concentration of nucleic acid inputs, as shown in FIG. 10. Only samples containing dsRNA (80 bp and ladder) were recognized. A 1 kb ssRNA that had been purified using cellulose (e.g., Baiersdörfer et al., Mol Ther Nucleic Acids. 2019 Feb. 27; 15:26-35) to remove dsRNA byproducts was only minimally detected at 100 ng. Synthetic single guide RNA (sgRNA) and linearized template DNA were not detected up to 200 ng.

### Example 6: Determination of Linear Range and Size Specificity of dsRNA Recognition by Av cGLR

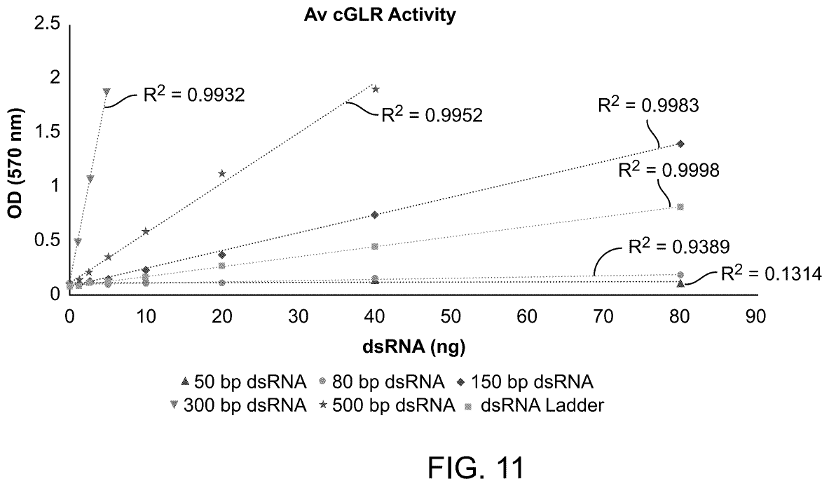

A serial dilution of dsRNA standards of different sizes (from 50-500 bp) and a ladder of several sizes (from 21-500 bp, NEB N0363) was prepared and combined with 300 nM Av cGLR, 1 mM ATP, 1 mM GTP, and 1×NEBuffer r2.1 in 50 μL reactions. The reactions were incubated at 37° C. for 60 minutes and quenched at 85° C. for 10 minutes. In a second step, the reactions were allowed to come to room temperature and were combined with 50 μL of the Kit manufacturer's pyrophosphate detection mix according to EXAMPLE 1. The final buffer composition of the combined reaction is 50% NEBuffer r2.1 and 50% of the recommended assay reaction buffer of EXAMPLE 1. The complete 100 μL reactions were transferred to a transparent 96-well plate and placed in a pre-warmed 37° C. SpectraMax M5 plate reader (Molecular Devices, San Jose, CA). Absorbance at 570 nm (OD (570 nm)) was measured continuously for 40 minutes. Using GraphPad Prism (GraphPad Software, San Diego, CA), the absorbance values at the 30 minute time point were plotted against the concentration of dsRNA inputs, as shown in FIG. 11. The 50 bp dsRNA was not detected up to 80 ng and the 80 bp dsRNA was only minimally detected at 80 ng. dsRNAs of 150-500 bp and the ladder reacted with OAS1 display a dose response. Linear regression analysis demonstrate a linear dose response for all detected samples, with the shortest range being 1.25-5 ng (300 bp) and the longest range being 1.25-80 ng (150 bp and ladder).

### Example 7: Estimation of dsRNA Amounts in In Vitro Transcribed RNAs Using Av cGLR

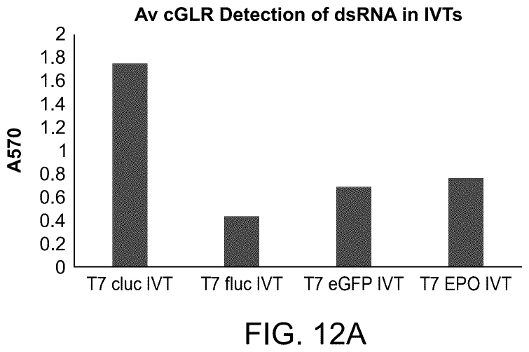

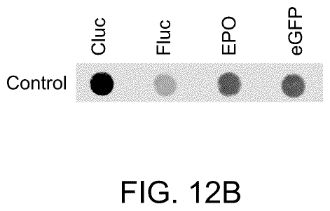

Cypridina Luciferase (CLUC), Firefly luciferase (FLUC), enhanced green fluorescent protein (EGFP), and erythropoietin (EPO) RNAs were prepared by IVT using appropriate template sequences, unmodified NTPs, and T7 RNA polymerase and then purified using the Monarch Spin RNA Cleanup Kit (NEB, Inc.) to remove carryover pyrophosphate. The total RNA concentration was quantified using a NanoDrop 8000 spectrophotometer (Thermo Scientific, Waltham, MA). A total of 1.5 μg of IVT RNA was assayed using the two-pot dsRNA detection reaction detailed in EXAMPLE 6. The absorbance values for all samples were plotted to assess relative amounts of dsRNA. FIG. 12A illustrates that the highest levels of dsRNA are detected in CLUC RNA. EGFP and EPO RNAs are comparable to each other and have substantially less dsRNA than CLUC RNA and FLUC RNA had the least amount of dsRNA of all RNAs tested. The same set of IVT RNAs were assayed by dot blot with J2 antibody and their relative amounts of dsRNA were assessed. Briefly, 1 μg of IVT RNA was spotted on Whatman nylon membrane (Sigma, #WHA10416294), air-dried for 20 min, UV crosslinked at 1200 mJ (×100) E settings, blocked for 1 hour at room temperature in 1×TBST+5% milk (Bio-Rad, #1706404), and incubated overnight at 4° C. with Scicons J2 dsRNA Antibody (Thermo, #10010200) diluted 1:5,000 in blocking buffer. The membrane was then washed three times with 1×TBST and incubated for 1 hour at room temperature with DyLight 800 4×PEG-conjugated secondary antibody (Cell Signaling Technologies, #5257P) diluted 1:10,000 in blocking buffer, washed three times with 1×TBST, and scanned using a Licor Odyssey infrared imaging system. As observed in FIG. 12B, the dot blot method results in the same pattern of relative dsRNA content between the four RNAs tested. As performed, the assay displays increasing intensity (increasing dsRNA) from lighter gray to darker gray. These data demonstrate the functionality of the Av cGLR and PPi detection assay as a method for the estimation of dsRNA present in IVT RNAs comparable to, but significantly faster than, dot blot assays.

What is claimed is:

1. A method comprising:
contacting an inactive dsRNA detector molecule and a dsRNA to produce an activated dsRNA detector molecule;
contacting the activated dsRNA detector molecule and a dsRNA detection substrate to produce a dsRNA detection intermediate;
contacting the dsRNA detection intermediate and a conversion detector to produce an activated conversion detector; and
contacting the activated conversion detector and a detection precursor to produce a detectable marker.

2. A method according to claim 1, wherein the activated dsRNA detector molecule comprises oligoadenylate synthase 1.

3. A method according to claim 1, wherein the dsRNA detection intermediate comprises pyrophosphate or an oligonucleotide.

4. A method according to claim 1, wherein the detectable marker indicates the presence of the dsRNA.

5. A method according to claim 1, wherein the dsRNA is included in a population of RNA molecules.

6. A method according to claim 5, wherein at least a portion of the population of RNA molecules are single-stranded.

7. A method according to claim 5, wherein the population of RNA molecules comprises at least one therapeutic RNA molecules.

8. A method according to claim 5, wherein the population of RNA molecules comprises one or more modified nucleotides.

9. A method according to claim 5, wherein the population of RNA molecules comprises one or more in vitro transcription products.

10. A method comprising:
contacting an inactive oligoadenylate synthase 1 (OAS1) and a dsRNA to produce pyrophosphate and an oligonucleotide; and
detecting the pyrophosphate or the oligonucleotide,
wherein the dsRNA is included in a population of RNA and the detected pyrophosphate and/or the detected oligonucleotide indicate that dsRNA is present in the population of RNA.

11. A method according to claim 10, wherein the population of RNA molecules comprises one or more modified nucleotides.

12. A method according to claim 10, wherein the population of RNA molecules comprises at least one therapeutic RNA molecules.

13. A method according to claim 10, wherein the population of RNA molecules comprises one or more in vitro transcription products.

14. A method according to claim 10, wherein the detecting the pyrophosphate or the oligonucleotide comprises detecting the pyrophosphate.

15. A method according to claim 14, wherein the detecting the pyrophosphate comprises contacting the pyrophosphate with a chemical detection reagent, wherein the chemical detection reagent comprises a metallochromic dye that can bind metals to form dye-metal complexes.

16. A method according to claim 15, wherein the chemical detection reagent is selected from 2-(5-bromo-2-pyridylazo)-5-[N-propyl-N-(3-sulfopropyl)amino]phenol; 2-(5-nitro-2-pyridylazo)-5-[N-propyl-N-(3-sulfopropyl)amino]phenol; 4-(2-pyridylazo) resorcinol; 2-(3,5-dibromo-2-pyridylazo)-5-(N-ethyl-N-sulfopropylamino)benzene; 2-(3,5-dibromo-2-pyridylazo)-5-[N-ethyl-N-(3-sulfopropyl)amino]phenol; and hydroxynaphthol blue.

17. A method according to claim 10, wherein the detecting the pyrophosphate comprises contacting the pyrophosphate with a chemical detection reagent that conditionally changes its absorption and/or fluorescence upon contact with or otherwise in the presence of pyrophosphate and detecting the change in absorption and/or fluorescence.

18. A method according to claim 10, wherein detecting the pyrophosphate or the oligonucleotide comprises detecting the pyrophosphate by contacting the pyrophosphate with a pyruvate phosphate dikinase under conditions that produce pyruvate and inorganic phosphate, contacting the produced pyruvate with a pyruvate oxidase under conditions that permit formation of hydrogen peroxide, and contacting the formed hydrogen peroxide with a peroxidase under conditions that permit conversion of 10-Acetyl-3,7-dihydroxyphenoxazine to 7-hydroxy-3H-phenoxazin-3-one.

19. A method according to claim 10, wherein detecting the pyrophosphate or the oligonucleotide comprises detecting the oligonucleotide by contacting the oligonucleotide with an RNase L under conditions that produce an active RNase L and contacting the active RNase L with a quenched single-stranded RNA reporter under conditions that permit cleavage of the reporter to yield a cleaved single-stranded RNA reporter.

20. A method comprising:
(a) contacting a dsRNA detector molecule with an RNA comprising or potentially comprising double-stranded RNA (dsRNA) to produce, if dsRNA is present, pyrophosphate and optionally, an oligonucleotide; and
(b) detecting the pyrophosphate or the oligonucleotide.

21. A method according to claim 20, wherein the dsRNA detector molecule comprises an oligoadenylate synthase or a cGLR.

22. A method according to claim 20, wherein the dsRNA detector molecule comprises an Asbolus verrucousus cGLR.

23. A method according to claim 20, wherein the detecting the pyrophosphate or the oligonucleotide comprises detecting the pyrophosphate.

24. A method according to claim 20, wherein the detecting the pyrophosphate comprises contacting the pyrophosphate with a chemical detection reagent, wherein the chemical detection reagent comprises a metallochromic dye that can bind metals to form dye-metal complexes.

25. A method according to claim 24, wherein the chemical detection reagent is selected from 2-(5-bromo-2-pyridylazo)-5-[N-propyl-N-(3-sulfopropyl)amino]phenol; 2-(5-nitro-2-pyridylazo)-5-[N-propyl-N-(3-sulfopropyl)amino]phenol; 4-(2-pyridylazo) resorcinol; 2-(3,5-dibromo-2-pyridylazo)-5-(N-ethyl-N-sulfopropylamino)benzene; 2-(3,5-dibromo-2-pyridylazo)-5-[N-ethyl-N-(3-sulfopropyl)amino]phenol; and hydroxynaphthol blue.

26. A method according to claim 20, wherein the detecting the pyrophosphate comprises contacting the pyrophosphate with a chemical detection reagent that conditionally changes its absorption and/or fluorescence upon contact with or otherwise in the presence of pyrophosphate and detecting the change in absorption and/or fluorescence.

27. A method according to claim 20, wherein detecting the pyrophosphate or the oligonucleotide comprises detecting the pyrophosphate by contacting the pyrophosphate with a pyruvate phosphate dikinase under conditions that produce pyruvate and inorganic phosphate, contacting the produced pyruvate with a pyruvate oxidase under conditions that permit formation of hydrogen peroxide, and contacting the formed hydrogen peroxide with a peroxidase under conditions that permit conversion of 10-Acetyl-3,7-dihydroxyphenoxazine to 7-hydroxy-3H-phenoxazin-3-one.

28. A method according to claim 20, wherein detecting the pyrophosphate or the oligonucleotide comprises detecting the oligonucleotide by contacting the oligonucleotide with an RNase L under conditions that produce an active RNase L and contacting the active RNase L with a quenched single-stranded RNA reporter under conditions that permit cleavage of the reporter to yield a cleaved single-stranded RNA reporter.

29. A method according to claim 20, wherein the dsRNA is included in a population of RNA molecules.

30. A method according to claim 29, wherein the population of RNA molecules comprises one or more fully single-stranded RNA molecules and/or one or more RNA molecules each comprising at least one single-stranded portion and at least one double-stranded portion.

31. A method according to claim 29, wherein the population of RNA molecules comprises one or more therapeutic RNA and/or one or more vaccine molecules.

32. A dsRNA detection kit comprising:
a dsRNA detector;
a chemical detection reagent; and
optionally, a buffer.

33. A dsRNA detection kit according to claim 32, wherein the dsRNA detector is selected from OAS1 and Av cGLR.

34. A dsRNA detection kit according to claim 32, wherein the chemical detection reagent is selected from 2-(5-bromo-2-pyridylazo)-5-[N-propyl-N-(3-sulfopropyl)amino]phenol; 2-(5-nitro-2-pyridylazo)-5-[N-propyl-N-(3-sulfopropyl)amino]phenol; 4-(2-pyridylazo) resorcinol; 2-(3,5-dibromo-2-pyridylazo)-5-(N-ethyl-N-sulfopropylamino)benzene; 2-(3,5-dibromo-2-pyridylazo)-5-[N-ethyl-N-(3-sulfopropyl)amino]phenol; and hydroxynaphthol blue.

35. A dsRNA detection kit according to claim 32 further comprising one or more dNTPs, one or more rNTPs, one or more polymerases, one or more kinases, one or more oxidases, one or more peroxidases, one or more RNase Ls, or combinations thereof.
